(19) United States
(12) Patent Application Publication
(10) Pub. No.: US 2024/0165282 A1
(43) Pub. Date: May 23, 2024

(54) CONFORMATIONAL CONSTRAINED SOMATOSTATIN RECEPTOR 3 PEPTIDE LIGANDS AND THEIR CONJUGATES AND USES THEREOF

(71) Applicant: STARGET PHARMA LTD., Ramat Hasharon (IL)

(72) Inventors: Michel AFARGAN, Ra'anana (IL); Eliav BLUM, Ginaton (IL); Yoseph SALITRA, Rehovot (IL)

(73) Assignee: STARGET PHARMA LTD., Ramat Hasharon (IL)

(21) Appl. No.: 18/548,898

(22) PCT Filed: Mar. 3, 2022
(86) PCT No.: PCT/IL2022/050238

§ 371 Date: Sep. 3, 2023

Related U.S. Application Data
(60) Provisional application No. 63/156,374, filed on Mar. 4, 2021.

(51) Int. Cl. A61K 51/08

(52) U.S. Cl. CPC A61K 51/088; A61K 2121/00; A61K 2123/00

(57) ABSTRACT

Embodiments of the invention relate to somatostatin receptor selective peptides to somatostatin receptor 3, methods for synthesizing the somatostatin analogs and pharmaceutical, and radiopharmaceutical compositions comprising somatostatin receptor 3 analogs, and methods of using such compositions are disclosed for treatment and for diagnosis.

### CROSS REFERENCE TO RELATED APPLICATIONS

Benefit is claimed to U.S. Provisional Patent Application 63/156,374, filed Mar. 4, 2021; the contents of which is incorporated by reference herein in its entirety.

### TECHNICAL FIELD

Embodiments of the invention relate to compositions comprising novel somatostatin receptor 3 analogs, and methods of using such compositions.

### BACKGROUND

Somatostatin, also known as growth hormone-inhibiting hormone (GHIH) or growth hormone release—inhibiting hormone (GHRIH) or somatotropin release—inhibiting factor (SRIF) is a naturally occurring inhibitory peptide hormone of 14 or 28 amino acid residues that regulates the endocrine system. It is secreted by the delta cells of the islets of the pancreas to inhibit the release of insulin and glucagon, and is also generated in the hypothalamus, where it inhibits the release of growth hormone (GH), adrenocorticotropic hormone (ACTH), prolactin and thyroid-stimulating hormones from the anterior pituitary. Somatostatin is initially secreted as a 116 amino acid precursor, preprosomatostatin, which undergoes endoproteolytic cleavage to prosomastatin. Prosomastatin is further processed into two active forms, somatostatin-14 (SST-14) and somatostatin-28 (SST-28), an extended SST-14 sequence to the N-terminus 1 (Bloom, S.R., and Polak, J.N. 1987). The actions of somatostatin are mediated via signaling pathways of G protein-coupled somatostatin receptors (GPCR). The somatostatin receptors (SSTR1, SSTR2, SSTR3, SSTR4 and SSTR5) belong to the G protein coupled receptor family and have a wide expression pattern in both normal tissues and diseased tissue or are overexpressed in varying conditions (Theodoropoulou M, et al, 2013; Møller LN, et al, 2003).

Antineoplastic effects and potential uses of somatostatin on various tumors, including pituitary adenomas, GEP-NETs (Gastroenteropancreatic neuroendocrine tumors), paragangliomas, carcinoids, breast cancers, malignant lymphoma and small-cell lung cancers, have been extensively investigated. Somatostatin has been used in the clinical setting for the palliative treatment (inhibition of excessive hormone release) and diagnosis of acromegaly and gastrointestinal tract tumors. The currently available somatostatin analogs octreotide and lanreotide have predominantly affinity for somatostatin receptor-2 (SSTR2) as agonists (Sun L, and Coy D.H., 2016; Reubi, J.C., et al, 2001; Hofiand, L.J., et al, 1994). Pasireotide is a somatostatin multi-receptor ligand with affinity for SSTR1, SSTR2, SSTR3 and SSTR5 and this broader binding profile may translate into a higher agonistic efficacy with respect to suppression of hormone release and cell growth in certain tumors (Anat Ben-Shlomo., et al, 2009). These analogs have been developed to achieve more favorable kinetics for efficiency use in the management of acute and chronic conditions, such as endocrine disorders associated with over secretion of hormones as well as upper gastrointestinal bleeding due to esophageal varices. Octreotide, which was the first approved somatostatin analog is a long-acting analog of somatostatin which exhibits nanomolar affinity and receptor specificity to SSTR2 that inhibits the release of several hormones and is clinically used to relieve symptoms of uncommon gastroentero-pancreatic endocrine tumors such as carcinoid, as well as to treat acromegaly which is associated with overexcretion of growth hormone. It is worth noting that the available nanomolar SSTR2 specific agonists Octreotide and Lanreotide enabled the elucidation of SSTR2 roles of somatostatin in endocrinology. These clinical available SSTR2 agonists demonstrate that SSTR2 is the specific receptor subtype which mediates the potent somatostatin inhibition of GH, ACTH, prolactin, insulin, glucagon, gastrin, vasoactive intestinal peptide, as well as the exocrine release of pancreatic lipase and amylase (Qian Z.R., et al, 2016).

In the early 1990's, the SSTR2 specific agonist octreotide was conjugated to DTPA and radiolabeled with Indium-111 which is [111In]In-DTPA-octreotide (111In-Pentetreotide™)—the first peptide-based radiopharmaceutical that was used as a diagnostic agent for scintigraphy SPECT imaging (Mikolajczak R, and Maecke H.R., 2016). Then, after a few years of clinical experience with [111In]In-pentetreotide, DOTA-chelated peptides, which could be more easily labeled with radioactive metals, started becoming available to be use with SPECT and PET imaging modalities (Kwekkeboom, D., et al, 2000). In the last decade there has been an exponential growth in the development of DOTA conjugated somatostatin analogs labeled with γ, β and α-emitting radionuclides for diagnostic as well as for therapeutic applications, mainly for NETs (Ansquer C, et al, 2009; Paganelli G, et al, 2001; Eychenne R., et al, 2020). The two most commonly used radiolabeled SSTR2 specific analogs, [⁹⁰Y]Y-DOTA-TOC and [¹⁷⁷Lu]Lu-DOTA-TATE, have demonstrated good clinical results with symptomatic relief, prolonged survival, and enhanced quality of patients life (Cives M., et al, 2017; Haider M., et al, 2020). This led to the approval of [¹⁷⁷Lu]Lu-DOTA-TATE (Lutathera™) in the treatment of SSTR2-positive (Peptide Receptor Radionuclide Therapy-PRRT) gastroenteropancreatic neuroendocrine tumors—GEP-NETs (Strosberg J., et al, 2021; Maqsood M.H., et al, 2019). SSTR-based theranostic radionuclide approach has been introduced in oncological application. Currently, theranostics (diagnostic established therapy) is a rapidly evolving method in an era of personalized and precision medicine.

It has been shown that several human malignancies of non-neuroendocrine origin including lung, breast and prostate cancer, also tend to over-express one or several SSTRs (Chin R.I., et al, 2021; Reubi J.C., et al, 2001). Therefore, efforts have been focused on the development of novel SST analogs with nanomolar affinity and specificity for different SSTRs subtypes.

### SUMMARY

Described herein is A somatostatin analog having the formula: R1-R2-DPhe-R3-Cys-R4-DTrp-Lys-Thr-R6-R5; wherein R¹ is an active agent, or is absent; R² is a linker, an active agent, or is absent; R³ is either Arg, Lys, or Orn; or optionally a polypeptide of 3 or two amino acids Glu-Glu-R⁷ or Glu-R⁷, wherein R⁷ is Arg, Lys, or Orn; R⁴ is either Phe or Tyr; and R⁵ is a NTAG having as structure of N-ThioAlkyl-Glycine

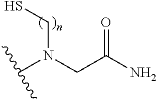 [SMILES: *N(CS)CC(N)=O]

wherein optionally a disulfide bond is formed between R⁵ and the cysteine residue and n is the number of methylene groups from 1 to 5; R⁶ is either Phe or Tyr; wherein when R³ is Arg, either R⁴ or R⁶ is Tyr or a pharmaceutically acceptable salt thereof.

Further described herein are diagnostic and therapeutic applications for the described somatostatin analog, in particular SEQ ID NO: 2, and its analogs for treatment and diagnosis of the described diseases and conditions.

Further described herein are methods for diagnosis and treatment of diseases associated with growth hormone, comprising administering SEQ ID NO:1 or SEQ ID NO: 2, and various derivatives thereof comprising active agents, to a patient in need thereof.

The foregoing and other objects, features, and advantages will become more apparent from the following detailed description, which proceeds with reference to the accompanying figures.

### BRIEF DESCRIPTION OF THE DRAWINGS

FIG. 1 is a comparative distribution profile of ⁶⁸Ga labeled SEQ ID NO: 1 (Arg²) and ⁶⁸Ga labeled SEQ ID NO: 1 (Lys²) in mice kidneys.

FIG. 2 is a comparative distribution profile of ⁶⁸Ga labeled SEQ ID NO: 1 (Arg²) and ⁶⁸Ga labeled SEQ ID NO: 1 (Lys²) in mice bladders.

FIG. 3 is a comparative distribution profile of ⁶⁸Ga labeled SEQ ID NO: 1 (Arg²) and ⁶⁸Ga labeled SEQ ID NO: 1 (Lys²) in mice heart.

FIG. 4 is a comparative endocrine profile of GH secretion for SEQ ID NO: 1 (Arg²) versus octreotide.

FIG. 5 is a comparative endocrine profile of GH secretion for SEQ ID NO: 2 (Lys²) versus octreotide.

FIG. 6 is a comparative endocrine profile of glucagon secretion for SEQ ID NO: 1 (Arg²) versus octreotide.

FIG. 7 is a comparative endocrine profile of glucagon secretion for SEQ ID NO: 2 (Lys²) versus octreotide;

FIG. 8 is a comparative endocrine profile of insulin secretion for SEQ ID NO: 1 (Arg²) and SEQ ID NO: 2 (Lys²) versus octreotide;

FIG. 9 shows the distribution profile of ⁶⁸Ga labeled SEQ ID NO: 1 (Arg²) in humans;

FIG. 10 shows the structural formula of SEQ ID NO: 1;

FIG. 11 shows the structural formula of SEQ ID NO: 2;

FIG. 12 shows the structural formula of SEQ ID NO: 3;

FIG. 13 shows the structural formula of SEQ ID NO: 15;

FIG. 14 shows the structural formula of SEQ ID NO: 16;

FIG. 15 shows the structural formula of SEQ ID NO: 17;

FIG. 16 shows the structural formula of SEQ ID NO: 18;

FIG. 17 shows the structural formula of SEQ ID NO: 27; and

FIG. 18 shows the structural formula of SEQ ID NO: 28.

### BRIEF DESCRIPTION OF THE DESCRIBED SEQUENCES

The nucleic and/or amino acid sequences provided herewith are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named 3296 1 2 SEQ LISTING_ST25 final-V001.txt, created Mar. 2, 2022, about 13.8 KB, which is incorporated by reference herein.

### DETAILED DESCRIPTION

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” “Consisting essentially of” indicates a composition, method, or process that includes only those listed features as the active or essential elements but can include non-active elements in addition. The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

Abnormal: Deviation from normal characteristics. Normal characteristics can be found in a control, a standard for a population, etc. For instance, where the abnormal condition is a disease condition, such as acromegaly, a few appropriate sources of normal characteristics might include an individual who is not suffering from the disease (e.g., progeria), a population standard of individuals believed not to be suffering from the disease, etc.

Administration: The introduction of a composition into a subject by a chosen route. Administration of an active compound or composition can be by any route known to one of skill in the art. Administration can be local or systemic. Examples of local administration include, but are not limited to, topical administration, subcutaneous administration, intramuscular administration, intrathecal administration, intrapericardial administration, intra-ocular administration, topical ophthalmic administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds and agents into implantable devices or constructs, such as vascular stents or other reservoirs, which release the active agents and compounds over extended time intervals for sustained treatment effects.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Agonist: A molecule or compound that binds to a target and stimulates a biological response, similar to the biological response of the native substance that normally binds to the target. The response may be inhibition or induction of cellular signals. Quantitatively the agonist response is defined by the value of effective concentration which elicits 50% of the maximal response which term as EC₅₀. Agonist can exhibit lower or higher EC₅₀ in comparison to the native substance. Targets can be receptors, proteins, transporters such as ions or nutrients channels, or enzymes. Agonists are not limited to a specific type of compound, and may include in various embodiments peptides, and fragments thereof, and other organic or inorganic compounds (for example, peptidomimetics and small molecules). Agonists may be endogenous, exogenous, full, partial, inverse, irreversible, or selective. Agonists may be superagnoist. Superagonists can exhibit similar, higher or lower EC₅₀ values relative to the native substance, but the intensity of the response elicited by a superagonist is significantly higher than the native substance.

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound. It is acknowledged that these terms may overlap in some circumstances.

Antagonist: A molecule or compound that tends to nullify the action of another, or in some instances that blocks the ability of a given chemical to bind to its receptor or other interacting molecule, preventing a biological response. Antagonists are not limited to a specific type of compound, and may include in various embodiments peptides, antibodies and fragments thereof, and other organic or inorganic compounds (for example, peptidomimetics and small molecules).

Autoimmune disease: A disease resulting from an aberrant immune response, such as the production of antibodies or cytotoxic T cells specific for a self-antigen or a subject's own cells or tissues. Autoimmune diseases include, but are not limited to, diabetes mellitus type 1, systemic lupus erythematosis , Churg-Strauss Syndrome, multiple sclerosis, Graves' disease, idiopathic thrombocytopenic purpura and rheumatoid arthritis, Hashimoto's autoimmune thyroiditis, Celiac disease, Vitiligo, Rheumatic fever, Pernicious anemia/atrophic gastritis, Addison disease, Dermatomyositis, Pernicious anemia, and Psoriasis.

Binding affinity: A term that refers to the strength of binding of one molecule to another at a site on the molecule. If a particular molecule will bind to or specifically associate with another particular molecule, these two molecules are said to exhibit binding affinity for each other. Binding affinity is related to the association constant and dissociation constant for a pair of molecules, but it is not critical to the methods herein that these constants be measured or determined. Rather, affinities as used herein to describe interactions between molecules of the described methods are generally apparent affinities (unless otherwise specified) observed in empirical studies, which can be used to compare the relative strength with which one molecule (e.g., an antibody or other specific binding partner) will bind two other molecules (e.g., two versions or variants of a peptide). The binding of a ligand to receptor can be determined by direct interaction of the labeled ligand to the receptor or alternatively by the displacement of the labeled native substance by the tested ligand. The binding studies disclosed herein represent the displacement approach which determine the concentration of the ligand which inhibits the interaction of the native substance to its receptor. Quantitatively the value used for these displacement determinations is the inhibitory concentration of the tested ligand needed for 50% reduction of interaction between the native substance and its receptor which termed as IC₅₀. The concepts of binding affinity, association constant, and dissociation constant are well known.

Binding domain: The molecular structure associated with that portion of a receptor that binds ligand. More particularly, the binding domain may refer to a polypeptide, natural or synthetic, or nucleic acid encoding such a polypeptide, the amino acid sequence of which represents a specific region (binding domain) of a protein, which either alone or in combination with other domains, exhibits binding characteristics. Neither the specific sequences nor the specific boundaries of such domains are critical, so long as binding activity is exhibited. Likewise, used in this context, binding characteristics necessarily includes a range of affinities, avidities and specificities, and combinations thereof, so long as binding activity is exhibited.

Cancer: The product of neoplasia is a neoplasm (a tumor or cancer), which is an abnormal growth of tissue that results from excessive cell division. A tumor that does not metastasize is referred to as “benign.” A tumor that invades the surrounding tissue and/or can metastasize is referred to as “malignant.” Neoplasia is one example of a proliferative disorder. A “cancer cell” is a cell that is neoplastic, for example a cell or cell line isolated from a tumor.

Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers (such as small cell lung carcinoma and non-small cell lung carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma, astrocytoma, medulloblastoma, craniopharyogioma, ependymoma, pinealoma, hemangioblastoma, acoustic neuroma, oligodendroglioma, meningioma, neuroblastoma and retinoblastoma).

Examples of neuroendocrine cancers, such as Adrenal cancer, Carcinoid tumors, Merkel cell carcinoma, Pancreatic neuroendocrine tumors, Paraganglioma, Pheochromocytoma, Medullary thyroid carcinoma, Pheochromocytoma of the adrenal gland, Small cell carcinoma, and Large cell carcinoid tumor.

Examples of vascular tumors, such as vascular tumors such as angiosarcoma, infantile hemangioma, congenital hemangioma, kaposiform hemangioendothelioma (KHE), and pyogenic granuloma.

Chelator: An agent useful in delivering metals into cells, which bind metal ions by chelation. Any suitable chelator may be used in implementing the teachings herein, for example but not limited to derivatives of DOTA (1,4,7,10-tetraazacyclododecane-1,4,7,10-tetraacetic acid), NOTA (2-(4,7-bis(2-(tert-butoxy)-2-oxoethyl)-1,4,7-triazonan- 1-yl) acetic acid), NODA (4-(4,7-bis(2-(tert-butoxy)-2-oxoethyl)-1,4,7-triazacyclononan-1-yl)-5-(tert-butoxy)-5-oxopentanoic acid) or EDTA (ethylenediaminetetraacetic acid). Additional chelators are described in WO03/006070. In some embodiments of the claimed invention the chelator is an anti-cancer compound.

Chemotherapeutic agent: An agent with therapeutic usefulness in the treatment of diseases characterized by abnormal cell growth or hyperplasia. Such diseases include cancer, autoimmune disease as well as diseases characterized by hyperplastic growth such as psoriasis. One of skill in the art can readily identify a chemotherapeutic agent (for instance, see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., © 2000 Churchill Livingstone, Inc; Baltzer L, Berkery R (eds): Oncology Pocket Guide to Chemotherapy, 2nd ed. St. Louis, Mosby-Year Book, 1995; Fischer DS, Knobf MF, Durivage HJ (eds): The Cancer Chemotherapy Handbook, 4th ed. St. Louis, Mosby-Year Book, 1993). Examples of chemotherapeutic agents include ICL-inducing agents, such as melphalan (Alkeran™), cyclophosphamide (Cytoxan™), cisplatin (Platinol™) and busulfan (Busilvex™, Myleran™). A chemotherapeutic agent may be used in combination with the described sequences.

Efficacy: Refers to the ability of agent to elicit a desired therapeutic effect. Efficacy also refers to the strength or effectiveness of a compound. As used herein, “enhancing efficacy” means to increase the therapeutic action of an agent. For example, when the agent is the described novel compounds, “enhancing efficacy” generally refers to increasing the ability of the agent to inhibit hormone secretion.

Effective amount of a compound: A quantity of compound sufficient to achieve a desired effect in a subject being treated. An effective amount of a compound can be administered in a single dose, or in several doses, for example daily, during a course of treatment. However, the effective amount of the compound will be dependent on the compound applied, the subject being treated, the severity and type of the affliction, and the manner of administration of the compound.

Functional fragments and variants of a polypeptide: Included are those fragments and variants that maintain one or more functions of the parent polypeptide. It is recognized that the gene or cDNA encoding a polypeptide can be considerably mutated without materially altering one or more the polypeptide's functions. First, the genetic code is well-known to be degenerate, and thus different codons encode the same amino acids. Second, even where an amino acid substitution is introduced, the mutation can be conservative and have no material impact on the essential functions of a protein. See Stryer, Biochemistry 3rd Ed., (c) 1988. Third, part of a polypeptide chain can be deleted without impairing or eliminating all of its functions. Fourth, insertions or additions can be made in the polypeptide chain for example, adding epitope tags, without impairing or eliminating its functions (Ausubel et al. Short Protocols in Molecular Biology, 4th ed., John Wiley & Sons, Inc., 1999). Other modifications that can be made without materially impairing one or more functions of a polypeptide include, for example, in vivo or in vitro chemical and biochemical modifications or the incorporation of unusual amino acids. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labeling polypeptides and labels useful for such purposes are well known in the art, and include radioactive isotopes such as 32P, ligands which bind to or are bound by labeled specific binding partners (e.g., antibodies), fluorophores, chemiluminescent agents, enzymes, and antiligands. Functional fragments and variants can be of varying length. For example, some fragments have at least 10, 25, 50, 75,100, or 200 amino acid residues.

Growth factor: a substance that promotes cell growth, survival, and/or differentiation. Growth factors include molecules that function as growth stimulators (mitogens), molecules that function as growth inhibitors (e.g. negative growth factors) factors that stimulate cell migration, factors that function as chemotactic agents or inhibit cell migration or invasion of tumor cells, factors that modulate differentiated functions of cells, factors involved in apoptosis, or factors that promote survival of cells without influencing growth and differentiation. Examples of growth factors are bFGF, EGF, CNTF, HGF, NGF, and actvin-A.

Growth Hormone: Also known as Somatotropin is an anabolic hormone which stimulates growth, cell reproduction, and cell regeneration. It is secreted by the anterior lobe of the pituitary gland and induces his growth effects via surface receptors expressed in tissue cells. In the liver, growth hormone stimulates the release of Insulin-Like-Growth-Factor-1 (IGF-1) which is the principal mediator of growth effects elicited by growth hormone in tissue cells.

Inhibiting protein activity: To decrease, limit, or block an action, function, or expression of a protein. The phrase inhibit protein activity is not intended to be an absolute term. Instead, the phrase is intended to convey a wide range of inhibitory effects that various agents may have on the normal (for example, uninhibited or control) protein activity. Inhibition of protein activity may, but need not, result in an increase in the level or activity of an indicator of the protein's activity. By way of example, this can happen when the protein of interest is acting as an inhibitor or suppressor of a downstream indicator. Thus, protein activity may be inhibited when the level or activity of any direct or indirect indicator of the protein's activity is changed (for example, increased or decreased) by at least 10%, at least 20%, at least 30%, at least 50%, at least 80%, at least 100% or at least 250% or more as compared to control measurements of the same indicator.

Inflammation: A localized protective response elicited by injury to tissue that serves to sequester the inflammatory agent. Inflammation is characterized by the appearance in or migration into any tissue space, unit, or region of any class of leukocyte in numbers that exceed the number of such cells found within such region of tissue under normal (healthy) circumstances. Inflammation is orchestrated by a complex biological response of vascular tissues to harmful stimuli, such as pathogens, damaged cells, or irritants. It is a protective attempt by the organism to remove the injurious stimuli as well as initiate the healing process for the tissue. An inflammatory response is an accumulation of white blood cells, either systemically or locally at the site of inflammation. The inflammatory response may be measured by many methods well known in the art, such as the number of white blood cells, the number of polymorphonuclear neutrophils (PMN), a measure of the degree of PMN activation, such as luminal enhanced-chemiluminescence, or a measure of the amount of cytokines present Inflammation can lead to a host of inflammatory diseases, such as but not limited to atherosclerosis, periodontitis, rheumatoid arthritis, Fatty liver disease, Endometriosis, Inflammatory bowel disease, glomerulonephritis Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process.

Label: A biomolecule attached covalently or noncovalently to a detectable label or reporter molecule. Typical labels include radioactive isotopes, such as but not limited to Gallium-68 (⁶⁸Ga), Lutitium-177 (¹⁷⁷Lu), Copper-64 (⁶⁴Cu), Indium-111 (¹¹¹In), Iodine-125 (¹²⁵I), Actinium-225 (²²⁵Ac), enzyme substrates, co-factors, ligands, chemiluminescent or fluorescent agents, haptens, and enzymes. Methods for labeling and guidance in the choice of labels appropriate for various purposes are discussed, for example, in Sambrook et al. (ed.), Molecular Cloning: A Laboratory Manual, 2nd ed., vol. 1-3, Cold Spring Harbor Laboratory Press, Cold Spring Harbor, NY, 1989 and Ausubel et al. Short Protocols in Molecular Biology, 4th ed., John Wiley & Sons, Inc., 1999. For example, ATP can be labeled in any one of its three phosphate groups with radioisotopes such as 32P or 33P, or in its sugar moiety with a radioisotope such as 35S.

Linker: One or more linear aliphatic chains or aromatic molecules or amino acids that serve as a spacer between two molecules, such as between two nucleic acid molecules or two peptides or between an agent, such as a chelator, or an active agent and the peptide. The linker, as a spacer, can improve the binding of the chelator-peptide conjugate by reduction of the steric effect of the chelator on the binding of the peptide ligand to the target receptor. The linker can act as pharmacokinetic agent by means of enhancer or reducer of renal clearance, plasma protein binding, distribution of the conjugate in human body or as enhancer of tumor uptake. A non-limited example of linkers are aromatic hydrophobic molecules such as phenylalanine or anionic amino acids such as aspartic or glutamic amino acids or aminomethyl benzoic acid, fatty acids, Near-infrared dyes such as Evan blue or fluorescein isothiocyanate (FITC) or, drugs such as acetaminophen, ibuprofen, warfarin, or other spacer molecules such as Gamma Amino Butyric Acid (GABA); 3-aminopropyltriethoxysilane (APTES); cross linkers such as: Diphenyl carbonate, diarylcarbonates, diisocyanates, pyromellitic anhydride, carbonyldiimidazole, epichloridrine, glutarldehyde, carboxylic acid dianhydrides, 2,2-bis(acrylamido) acetic acid, and dichloromethane. Any suitable linker may be used in implementing the teachings herein, for example, linkers such as described in Publications AU729225, US2004/0166499, U.S. Pat. No. 5,854,194, U.S. Pat. No. 6,303,555, WO2017/066668, U.S. Pat. No. 6,297,191, U.S. Pat. No. 6,020,301, or US 2010/0240773.

Marker: A protein, or a gene encoding a protein, for which a system is available to identify cells that produce the protein. In one specific, non-limiting example of a selectable marker is a protein, or a gene encoding a protein, that can be identified in a cell based on its fluorescent or enzymatic properties. Specific, non-limiting examples include, but are not limited to, fluorescent marker fluorescein isothiocyanate (FITC), enhanced green fluorescent protein (EGFP), alkaline phosphatase, or horseradish peroxidase. A marker can also be a polypeptide or antigenic epitope thereof, wherein an antibody that specifically binds the polypeptide can be used to identify cells that express the polypeptide or antigenic epitope. One specific, non-limiting example of a polypeptide of use is human growth hormone (hGH). Additional specific non-limiting examples of a marker include drug resistance markers, such as G148 or hygromycin. Additionally, a marker can be a protein or a gene encoding a protein for which negative selection can be used to identify the cell expressing the marker. A specific, non-limiting example of a negative selection marker includes, but is not limited to, the HSV-tk gene. This gene will make the cells sensitive to agents such as acyclovir and gancyclovir. Another specific, non-limiting example of a selectable marker is a protein, or a gene encoding a protein, wherein selection can be made by using a cell surface marker, for example, to select over-expression of the marker by fluorescence activated cell sorting (FACS).

Pharmaceutically acceptable carriers: The pharmaceutically acceptable carriers useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of the compounds herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or composition capable of inducing a desired therapeutic or prophylactic effect when properly administered to a subject or a cell. Incubating includes exposing a target to an agent for a sufficient period of time for the agent to interact with a cell. Contacting includes incubating an agent in solid or in liquid form with a cell.

Polypeptide: A polymer in which the monomers are amino acid residues which are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

The term polypeptide fragment refers to a portion of a polypeptide which exhibits at least one useful epitope. The phrase “functional fragments of a polypeptide” refers to all fragments of a polypeptide that retain an activity, or a measurable portion of an activity, of the polypeptide from which the fragment is derived. Fragments, for example, can vary in size from a polypeptide fragment as small as an epitope capable of binding an antibody molecule to a large polypeptide capable of participating in the characteristic induction or programming of phenotypic changes within a cell. An epitope is a region of a polypeptide capable of binding an immunoglobulin generated in response to contact with an antigen.

Preventing or treating a disease: Preventing a disease refers to inhibiting the full development of a disease, for example inhibiting the development of myocardial infarction in a person who has coronary artery disease or inhibiting the progression or metastasis of a tumor in a subject with a neoplasm. Treatment refers to a therapeutic intervention that ameliorates a sign or symptom of a disease or pathological condition after it has begun to develop.

Radiation Therapy (Radiotherapy): The treatment of disease (e.g., cancer or another hyperproliferative disease or condition) by exposure of a subject or their tissue to a radioactive substance. Radiation therapy is the medical use of ionizing radiation as part of cancer treatment to control malignant cells. Radiotherapy may be used for curative or adjuvant cancer treatment. It is used as palliative treatment where cure is not possible and the aim is for local disease control or symptomatic relief. Radiation therapy may be used in combination with the described sequences.

Senescence: Refers to the essentially irreversible growth arrest that occurs when cells that can propagate stop dividing and is often referred to as just “senescence.” Cellular senescence was formerly described as a process that reduces the proliferation (growth) of normal human cells in culture. There are numerous senescence-inducing stimuli. Furthermore, many senescent cells harbor genomic damage at non-telomeric sites, which also generate the persistence of DNA damage signaling needed for the senescence growth arrest. DNA double strand breaks are especially potent senescence inducers. The senescence growth arrest is not simply a halt in cell proliferation. Senescent cells show marked and distinct changes in their pattern of gene expression.

The biological process(es) of aging and showing the effects of increased age. In one embodiment, a senescent cell does not divide and/or has a reduced capacity to divide, which may be a cause of male infertility.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of a Y-family polymerase protein, and the corresponding cDNA sequence, will possess a relatively high degree of sequence identity when aligned using standard methods. This homology will be more significant when the orthologous proteins or cDNAs are derived from species which are more closely related (e.g., human and chimpanzee sequences), compared to species more distantly related (e.g., human and C. elegans sequences).

The NCBI Basic Local Alignment Search Tool (BLAST) (Altschul et al. J. Mol. Biol. 215:403-10, 1990) is available from several sources, including the National Center for Biotechnology Information (NCBI, Bethesda, MD) and on the Internet, for use in connection with the sequence analysis programs blastp, blastn, blastx, tblastn and tblastx. It can be accessed at the NCBI website, together with a description of how to determine sequence identity using this program.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tm) for the specific sequence at a defined ionic strength and pH. The Tm is the temperature (under defined ionic strength and pH) at which 50% of the target sequence remains hybridized to a perfectly matched probe or complementary strand. Conditions for nucleic acid hybridization and calculation of stringencies can be found in Sambrook et al. (ed.), Molecular Cloning: A Laboratory Manual, 2nd ed., vol. 1-3, Cold Spring Harbor Laboratory Press, Cold Spring Harbor, NY, 1989, and Tijssen Laboratory Techniques in Biochemistry and Molecular Biology—Hybridization with Nucleic Acid Probes Part I, Chapter 2, Elsevier, New York, 1993.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, NY, 1989, chapters 9 and 11, herein incorporated by reference. The following is an exemplary set of hybridization conditions:

### Very High Stringency (Detects Sequences that Share 90% Identity)

Hybridization: 5× SSC at 65° C. for 16 hours
Wash twice: 2× SSC at room temperature (RT) for 15 minutes each
Wash twice: 0.5× SSC at 65° C. for 20 minutes each

### High Stringency (Detects Sequences that Share 80% Identity or Greater)

Hybridization: 5×-6× SSC at 65° C.-70° C. for 16-20 hours
Wash twice: 2× SSC at RT for 5-20 minutes each
Wash twice: 1× SSC at 55° C.-70° C. for 30 minutes each

### Low Stringency (Detects Sequences that Share Greater than 50% Identity)

Hybridization: 6× SSC at RT to 55° C. for 16-20 hours
Wash at least twice: 2×-3× SSC at RT to 55° C. for 20-30 minutes each.

Nucleic acid sequences that do not show a high degree of identity can nevertheless encode similar amino acid sequences, due to the degeneracy of the genetic code. It is understood that changes in nucleic acid sequence can be made using this degeneracy to produce multiple nucleic acid molecules that all encode substantially the same protein.

Specifically, hybridizable and specifically complementary are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Somatostatin: An inhibitory hormone with mainly neuroendocrine inhibitory effects.

Somatostatin receptor: There are five main somatostatin receptors, SSTR1-SSTR5. The expression of the SSTRs has been noted to be varied throughout the body: SSTR1 is expressed in highest levels in the jejunum and stomach; SSTR2 is expressed in highest levels in the cerebrum, pituitary, pancreas, intestines and kidney; SSTR3 is expressed in highest levels in the brain and testis; SSTR4 is expressed in highest levels in the fetal and adult brain and lungs; and SSTR5 is expressed in highest levels in the brain, pituitary gland, pancreas (alpha and gamma cells) as well as in the gastrointestinal tract. SSTRs are also overexpressed in some pathological cells, including many different types of cancerous cells or result in conditions such as acromegaly.

Subject: Living multi-cellular organisms, including vertebrate organisms, a category that includes both human and non-human mammals.

Superagonist: A ligand that is capable of producing a maximal response in the target receptor greater than the endogenous agonist. The response can be activation or inhibition of cellular signals such as, but not limited, enzymatic activity, ions channels, transporters, proteins, secretory of cellular hormones or enzymes.

Therapeutically effective amount: A quantity of compound sufficient to achieve a desired effect in a subject being treated. An effective amount of a compound may be administered in a single dose, or in several doses, for example daily, during a course of treatment. However, the effective amount will be dependent on the compound applied, the subject being treated, the severity and type of the affliction, and the manner of administration of the compound. For example, a therapeutically effective amount of an active ingredient can be measured as the concentration (moles per liter or molar-M) of the active ingredient (such as a small molecule, peptide, protein, or antibody) in blood (in vivo) or a buffer (in vitro) that produces an effect.

### II. Overview of Several Embodiments

Despite the well-established clinical role of SSTR2 and its available nanomolar specific ligands in somatostatin pharmacology, the specific endocrine role of other SSTR subtypes SSTR1, SSTR3, SSTR4 and S STR5 remained unknown. SSTR3 is the somatostatin receptor subtype with the highest molecular size, highest intensity of internalization, and the only receptor associated with substantial role in cell-cycle arrest and apoptosis among the other SSTRs. These differentiated properties of SSTR3 make this receptor subtype as an attractive target in endocrinology and cancer. Although, a recent work reported on the first successful development of peptide-based SSTR3 agonists, the reported analogs do not elicit sufficient nanomolar affinity to SSTR3, and they elicit antiproliferative effect on non-functional pituitary adenoma, but their endocrine effects of hormone release were not elucidated (Vazquez-Borrego et al. 2019).

Described herein are receptor specific superagonists of SSTR3 and methods of using them, in particular relating to their in vivo endocrine effect on GH release. Moreover, these SSTR3 agonists elicit high selectivity to the pituitary GH release but not on pancreatic release of insulin and glucagon. These novel and unexpected findings show for the first time that SSTR3 specific agonists mediate via SSTR3 the somatostatin inhibition of GH release. For example, the SSTR3 superagonist SEQ ID NO: 4 was evaluated for off-target interactions against more than 167 human cloned GPCRs and 44 human cloned pharmacological targets. The data confirmed high affinity and super-selectivity to human SSTR3 while no binding of clinical significance indicated for all other (n=211) human targets. This data supports that the unexcepted in vivo results of inhibition of GH release is solely via SSTR3. Furthermore, the conjugation of these novel SSTR3 agonists to chelators such as DOTA did not interfere with their binding affinity and selectivity to SSTR3 making them as potential superagonist and superselective candidates for radioactive diagnosis and treatment of endocrine syndromes such as acromegaly, a pituitary adenoma associated with over secretion of GH, as well as in various malignancies that might be associated with overexpression of SSTR3.

### Active Agent

In certain embodiments of the claimed invention, the somatostatin may comprise an active agent. The active agent may covalently bonded to the cyclic peptide such as set forth in SEQ ID NO: 1 and SEQ ID NO: 2. The active agent may be an imaging moiety, a therapeutic moiety, a dye, a fluorescent moiety, a toxin, a chelator, a double chelator linked by a Lysine amino acid, a moiety with a metal atom, a moiety with a radioactive atom, a nanoparticle, an ethylene glycol polymer, a photosensitizer, a liposome constituent and a micelle constituent, a chelator covalently bound to moiety which increases the hydrophobicity and plasma protein binding of the peptide conjugate such as the dye Evans blue, or the drug ibuprofen. In some embodiments of the invention a single active agent moiety falls within the definition of two or more elements of the above group, for example: in some embodiments an active agent moiety is a nanoparticle and an ethylene glycol polymer and either or both of a therapeutic/imaging agent moiety; in some embodiments an active agent moiety is a dye, and/or fluorescent moiety and/or a photosensitizer and either or both of a therapeutic/imaging agent moiety; in some embodiments an active agent moiety includes a radioactive atom and falls within one or more of the other definitions.

In some embodiments, such an active agent moiety is bonded directly to the N-terminal amino acid. In some embodiments, such an active agent moiety is bonded indirectly to the N-terminal amino acid, e.g., through a linker (e.g., GABA (gamma-aminobutyric acid), an amino acid, a peptide chain).

### Size of Active Agent Moiety

The size of the active agent moiety is any suitable size. That said, in some embodiments, the active agent moiety has a molecular weight of not less than 250, not less than 500, not less than 750, not less than 1000, not less than 2000, not less than 4000, not less than 8000, and even not less than 16000.

### Imaging Moiety

In some embodiments, the active agent moiety is an imaging moiety, that is to say, is an agent that is distinctly observable when concentrated in a cell under suitable conditions and/or when using a suitable imaging modality.

Any suitable imaging moiety may be used in implementing the teachings herein. Typical imaging moieties include moieties having a distinct color allowing visual identification, moieties having distinct fluorescence allowing visual identification under appropriate lighting conditions, or positron emitters allowing imaging by Positron Emission Tomography.

In some such embodiments, subsequent to administration of the Somatostatin receptor ligand to cells, the imaging moieties become concentrated on the surface and within the cells expressing Somatostatin receptor to a greater degree than others, allowing identification of such cells. A typical utility of such embodiments is to differentiate between normal cells and pathological cells overexpres sing Somatostatin receptors.

### Therapeutic Moiety

In some embodiments, the active agent moiety is a therapeutic moiety, when concentrated in a cell the active agent moiety has some desired pharmacological effect, typically including helping a targeted cell develop or attenuating growth of or killing a targeted cell, for example when the targeted cell is pathological.

Any suitable therapeutic moiety may be used in implementing the teachings herein, for example, a toxin, a vitamin, and a photosensitizer. Typical therapeutic moieties include moieties that are cytotoxic when concentrated in a cell, for example by influencing cell processes or free radical or radiation damage.

In some such embodiments, subsequent to administration of the Somatostatin receptor ligand to cells, the therapeutic moieties become concentrated on the surface and within the cells expressing Somatostatin receptors (e.g., especially cells overexpressing Somatostatin receptors), to a greater degree than cells not expressing Somatostatin receptors or cells expressing low levels of Somatostatin receptors allowing specific targeting and treatment of such cells. A typical utility of such embodiments is to administer a cell-killing active agent to pathological cells overexpressing Somatostatin receptors (e.g., some cancers) while causing little or no damage to normal cells.

### Dye

In some embodiments, the active agent moiety is a dye, an active agent moiety that includes a chromophore having a distinct color that can be observed at sufficient concentration.

Any suitable dye with any suitable chromophore may be used in implementing the teachings herein, for example, derivatives of methyl violet.

In some such embodiments, subsequent to administration of the Somatostatin receptor ligand to cells (in vivo or in vitro), the dyes become concentrated in cells expressing Somatostatin receptors to a greater degree than others (e.g., especially cells overexpressing Somatostatin receptors), allowing identification of such cells by visual or microscopic inspection. In some embodiments the dye can be used to increase the hydrophobicity and plasma protein binding of the peptide conjugate.

### Fluorescent Agent

In some embodiments, the active agent moiety is fluorescent, an active agent moiety that includes a fluorophore that absorbs energy at a first wavelength of light, and then emits at least some of the energy at a second wavelength of light higher than the first.

Any suitable fluorescent agent with any suitable fluorophore may be used in implementing the teachings herein, for example, derivatives of fluorescein or rhodamine.

In some such embodiments, subsequent to administration of the Somatostatin receptor ligand to cells (in vivo or in vitro), the fluorescent agents become concentrated in cells expressing Somatostatin receptors to a greater degree than others (e.g., especially cells overexpressing Somatostatin receptors), allowing identification of such cells due to the distinct fluorescence of the fluorophore.

### Toxin

In some embodiments, the active agent moiety is a toxin, an active agent that has a deleterious effect on cells, for example, by disrupting biological processes in the cell, for example by attenuating or stopping cell development (e.g., cytostatic) or even killing the cell (e.g., cytotoxic).

In some such embodiments, subsequent to administration of the Somatostatin receptor ligand to cells, the toxin becomes concentrated in cells expressing Somatostatin receptors to a greater degree than others, e.g., especially cells overexpressing Somatostatin receptors, thereby having a deleterious effect on the cells.

Any suitable toxin may be used in implementing the teachings herein, for example, derivatives of actinomycin, camptothecin, doxorubicin, gentamicin. Some such embodiments can be used in vivo to damage or kill cells overexpressing one or more Somatostatin receptor.

### Chelator

In some embodiments, the active agent moiety is a chelator, an active agent configured to bind metal ions by chelation. Any suitable chelator may be used in implementing the teachings herein, for example derivatives of DOTA (1,4,7,10-tetraazacyclododecane-1,4,7,10-tetraacetic acid), NOTA (2-(4,7-bis(2-(tert-butoxy)-2-oxoethyl)-1,4,7-triazonan-1-yl) acetic acid), NODA (4-(4,7-bis(2-(tert-butoxy)-2-oxoethyl)-1,4,7-triazacyclononan-1-yl)-5-(tert-butoxy)-5-oxopentanoic acid) or EDTA (ethylenediaminetetraacetic acid) DTPA (Diethylenetriamine pentaacetate). In some such embodiments, the active agent moiety is a chelator that is chelating a metal, in some embodiments a radioactive or MRI detectable metal, as described below. The chelator may or may not have the metal ion bound to it.

In some embodiments, subsequent to administration of the Somatostatin receptor ligand to cells, the chelator (depending on the embodiment, with or without chelated metal) become concentrated in cells expressing one or more Somatostatin receptors to a greater degree than others, e.g., especially cells overexpressing Somatostatin receptors.

Some such embodiments can be used to concentrate metal ions (in some embodiments MRI-detectable metals, or/and radioactive metal ions) in cells overexpres sing one or more Somatostatin receptor, e.g., for imaging and/or therapeutic purposes.

Moiety with a Metal Atom

In some embodiments, the active agent moiety is a moiety with a metal atom. Some such embodiments can be used to concentrate metal atoms in cells overexpressing Somatostatin receptors.

For example, in some embodiments the metal atom is a radioactive metal atom, and the Somatostatin is for use as a radiopharmaceutical in the field of nuclear medicine, e.g., for therapeutic and/or imaging purposes, as described below.

For example, in some embodiments the metal atom is an MRI-detectable metal atom (e.g., Gadolinium, Fe2+) for use as an MRI contrasting agent in the field of magnetic resonance imaging, e.g., for imaging purposes.

Moiety with Radioactive Atom

In some embodiments, the active agent moiety is a moiety with a radioactive atom. Some such embodiments can be used to concentrate radioactive atoms in cells overexpressing one or more Somatostatin receptor, for example, for use as a radiopharmaceutical in the field of nuclear medicine, e.g., for therapeutic and/or imaging purposes.

In some such embodiments, subsequent to administration of the Somatostatin receptor ligand to cells, the radioactive atoms become concentrated in cells expressing one or more Somatostatin receptor to a greater degree than others (e.g., especially cells overexpressing one or more Somatostatin receptors), allowing identification of such cells with radiation-detecting moieties (e.g., PET/SPECT) and/or having a therapeutic (e.g., toxic) effect due to emitted radiation.

Any suitable moiety with any suitable radioactive agent may be used in implementing the teachings herein.

In some embodiments, the radioactive atom is covalently bonded to other parts of the active agent moiety. Typical such embodiments include one or more radioactive atoms, for example, atoms selected from the group consisting of iodine-123, iodine-125, iodine-131 in an iobenguane residue, fluorine-18, carbon-11, carbon-14, tritium, nitrogen-13, oxygen-15 and phosphorous-32.

In some embodiments, the radioactive agent is a radioactive metal atom ionically bonded (e.g., chelated) to other parts of the active agent moiety. Typical such embodiments include one or more radioactive atoms, for example, atoms selected from the group consisting of actinium-225, bismuth- 213, technetium-99m, chromium-51, cobalt-57, cobalt-58, copper-64, erbium-169, gallium-67, gallium-68, indium-111, iron-59, lutetium-177, radium-223, rubidium-82, samarium-153, selenium-75, strontium-89, thallium-201 and yttrium-90.

### Nanoparticle

In some embodiments, the active agent moiety comprises a nanoparticle, and in some embodiments is a nanoparticle. A person having ordinary skill in the art is familiar with the definition of the term “nanoparticle”, see for example, Murthy SK, Int J Nanomedicine 2007, 2(2) 129-141 which is included by reference as if fully set-forth herein, that also includes examples of specific nanoparticles that may be used in implementing the teachings herein. That said, in some embodiments, a nanoparticle is a particle of not less than 1 nanometer in size and not more than 1000 nanometers in size, and in some embodiments not more than 100 nanometers in size.

In some such embodiments, subsequent to administration of the Somatostatin receptor ligand to cells, the nanoparticles moieties become concentrated in cells expressing one or more Somatostatin receptor to a greater degree than others (e.g., especially cells overexpressing one or more Somatostatin receptor), allowing identification of such cells.

In some embodiments, the nanoparticle defines an internal volume containing a secondary active agent (e.g., a therapeutic or imaging agent). In some such embodiments, the nanoparticle is used as a container for delivery of the secondary active agents contained therein into a cell. Some such embodiments are used to deliver large amounts of the secondary active agents to cells overexpressing one or more Somatostatin receptors: once the nanoparticle is internalized in a cell, the secondary active agent is released inside the cell. In some such embodiments, subsequent to administration of the Somatostatin receptor ligand to cells, the nanoparticles moieties become concentrated in cells expressing one or more Somatostatin receptor to a greater degree than others (e.g., especially cells overexpressing one or more Somatostatin receptor), and then release the secondary active agent inside the cell.

Any suitable nanoparticle may be used in implementing the teachings herein, for example, nanoparticles such as described in PCT Publications WO2012/054923, WO2012/166923, WO2014/04361 and WO2014/043625 which are included by reference as if fully set-forth herein, as well as nanoparticles that are substantially albumin clusters.

In some embodiments, the nanoparticle comprises carbon nanotubes, especially single-walled carbon nanotubes. In some embodiments, the carbon nanotubes are (optionally fluorinated and then) modified with branched polyethyleneimine through which the cyclic peptide moiety is covalently bonded. In some embodiments, the carbon nanotube further comprises a therapeutic active agent bonded to the carbon nanotube, for example, through a branched polyethyleneimine. Such embodiments may be implemented by a person having ordinary skill in the art upon perusal of the specification in combination with the teachings of Andreoli E et al, in J. Mater. Chem. B 2014, 2, 4740-4747, which is included by reference as if fully set forth herein.

In some embodiments, the nanoparticle comprises a “nanoflower”, for example, formed of a graft copolymer constructed by directly polymerizing gamma-camptothecin-glutamate N-carboxyanhydride (Glu(CPT)-NCA) on multiple sites of poly(ethylene glycol) (PEG)-based main chain via ring open polymerization (ROP). Such embodiments may be implemented by a person having ordinary skill in the art upon perusal of the specification in combination with the teachings of Tai W et al, in J. Biomaterials 2014, 35(25), 7194-7203 which is included by reference as if fully set forth herein.

### Ethylene Glycol Polymer

In some embodiments, the active agent moiety comprises an ethylene glycol polymer (polyethylene glycol). In some such embodiments, the ethylene glycol polymer is a component of a nanoparticle.

In some such embodiments, the peptide moiety of the Somatostatin receptor ligand as described herein is pegylated by the ethylene glycol polymer. Depending on the embodiment, pegylation may have one or more useful attributes including increasing solubility (in vivo and/or in vitro), reducing in vivo immunogenicity and antigenicity, and reducing the rate of renal clearance of the Somatostatin receptor ligand.

### Photosensitizer

In some embodiments, the active agent moiety is a photosensitizer. A photosensitizer is a molecule that absorbs energy from light to enter an excited state, and in the excited state interacts with triplet oxygen species to produce chemically active singlet oxygen species. Known photosensitizers include phenothiazines such as Methylene Blue, xanthenes like Rose Bengal and porphyrins.

In some such embodiments, subsequent to administration of the Somatostatin receptor ligand to cells, the photosensitizer moieties become concentrated in cells expressing Somatostatin receptors to a greater degree than others (e.g., especially cells overexpressing one or more Somatostatin receptor). Once the photosensitizers are inside the cell, the cells are irradiated, causing the photosensitizers to generate active oxygen species inside the cell from oxygen molecules present inside the cell, the active oxygen species having a potential cytotoxic effect.

### Liposome Constituent

In some embodiments, the active agent moiety is a liposome constituent, e.g., a phospholipid or an ethylene glycol polymer. In some such embodiments, the Somatostatin receptor ligand is used to form a liposome together with other liposome constituents (as known in the art) optionally with secondary active agent contained inside the liposome in analogy to the described with reference to nanoparticles above. In such embodiments, the liposome-constituent active-agent moiety becomes part of the liposome while at least part of the peptide moiety acts as a guiding moiety to preferentially or even selectively bind the liposome to cells that express or overexpress Somatostatin receptors. Some such embodiments are used to deliver liposomes (and in some embodiments, secondary active agents contained therein) into cells overexpressing one or more Somatostatin receptor.

In some such embodiments, subsequent to administration of the Somatostatin receptor ligand to cells, the liposome become concentrated in cells expressing one or more Somatostatin receptors to a greater degree than others (e.g., especially cells overexpressing one or more Somatostatin receptors), and then release the secondary active agent inside the cell.

### Micelle Constituent

In some embodiments, the active agent moiety is a micelle constituent, e.g., a surfactant. In some such embodiments, the Somatostatin receptor ligand is used to form a micelle together with other micelle constituents (as known in the art) optionally with secondary active agent contained inside the micelle in analogy to the described with reference to nanoparticles and liposomes above. In such embodiments, the micelle-constituent active-agent moiety becomes part of the micelle while at least part of the peptide moiety acts as a guiding moiety to preferentially or even selectively bind the micelle to cells that express or overexpress one or more Somatostatin receptor. Some such embodiments are used to deliver micelles (and in some embodiments, secondary active agents contained therein) into cells overexpres sing one or more Somatostatin receptor.

In some such embodiments, subsequent to administration of the Somatostatin receptor ligand to cells, the micelle become concentrated in cells expressing one or more Somatostatin receptor to a greater degree than others (e.g., especially cells overexpressing one or more Somatostatin receptor), and then release the secondary active agent inside the cell.

### Tumor Targeting Agents

In some embodiments, the active agent moiety is a tumor targeting agent. In particular embodiments, the conjugation is to amino acid sequences that are not somatostatin but target other targets to enhance the binding of the somatostatin to tumors such as but not limited to, tripeptide Arg-Gly-Asp (RGD) which belongs to the family of integrins that are used as a tumor adhesive motifs.

In some embodiments the conjugation of the somatostatin sequence is to a lipid moiety such as hexanoic acid, heptanoic acid, octanoic acid (caprylic acid), decanoic acid, myristic acid, lauric acid, palmitic acid, oleic acid, linoleic acid to increase the peptide bioavailability to specific tissues such as tumors. In some embodiments R² is at least one tumor targeting agent.

### Linker

In some embodiments R² is a linker. Non-limited examples of linkers are aromatic hydrophobic molecules such as phenylalanine or anionic amino acids such as aspartic or glutamic amino acids or aminomethyl benzoic acid, fatty acids, Near-infrared dyes such as Evan blue or fluorescein isothiocyanate (FITC) or, drugs such as acetaminophen, ibuprofen, warfarin, or other spacer molecules such as Gamma Amino Butyric Acid (GABA); Para Aminobenzoic Acid (PABA), 4-aminomethyl-benzoic acid, 8-aminooctanoic acid, 3-aminopropyltriethoxy silane (APTES); cross linkers such as: Diphenyl carbonate, diarylcarbonates, diisocyanates, pyromellitic anhydride, carbonyldiimidazole, epichloridrine, glutarldehyde, carboxylic acid dianhydrides, 2,2-bis(acrylamido) acetic acid, and dichloromethane. In some embodiments the linker is a lipid or amino-lipid moiety such as hexanoic or amino hexanoic acid, heptanoic or amino heptanoic acid, octanoic (caprylic) or amino octanoic acid, decanoic or amino decanoic acid, myristic or amino myristic acid, lauric or amino lauric acid, palmitic or amino palmitic acid, oleic or amino oleic acid, linoleic or amino linoleic acid which may improve the binding with the a chelator such as DOTA or to increase the peptide bioavailability to specific tissues such as tumors.

A linker may be an amino acid or a polypeptide, preferably between 1 and 7 amino acids in length, most preferably between 1 and 3 amino acids in length. The amino acid preferred as a linker is selected from the group of one or more than one of: Gly, Ala, Lys, and Phe.

Any suitable linker may be used in implementing the teachings herein, for example, linkers such as described in Publications AU729225, US2004/0166499, U.S. Pat. No. 5,854,194, U.S. Pat. No. 6,303,555, WO2017/066668, U.S. Pat. No. 6,297,191, U.S. Pat. No. 6,020,301, or US 2010/0240773.

In other particular embodiments, the pharmaceutical composition can further include a pharmaceutically acceptable carrier, diluent, or salt, all of which are standard and known in the art.

### Exemplary Compositions

Some of the compositions (somatostatin analogs) described herein have the following general formulae, R¹-R²-DPhe-R³-Cys-R⁴-DTrp-Lys-Thr-R⁶-R⁵ wherein R1, R2, R3, R4, and R6 are defined as in Table 1 below and R⁵ is NTAG having a structure of N-ThioAlkyl-Glycine

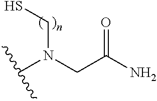

wherein a disulfide bond is formed between R⁵ and the cysteine residue (located between R³ and R⁴) or a pharmaceutically acceptable salt thereof, and n is 2. (-) indicates that the substituent is absent. “4-Amb” stands for 4-aminomethyl-benzoic acid. “Orn” stands for ornitihine. “CPT” stands for camptothecin. “GABA” stands for gamma-aminobutyric acid (C₄H₉NO₂).

TABLE 1

| Sequence | | | | | |
|---|---|---|---|---|---|
| ID No. | R¹ | R² | R³ | R⁴ | R⁶ |
| 1 | — | — | Arg | Phe | Phe |
| 2 | — | — | Lys | Phe | Phe |
| 3 | DOTA | — | Arg | Phe | Phe |
| 4 | Ga-DOTA | — | Arg | Phe | Phe |
| 5 | Cu-DOTA | — | Arg | Phe | Phe |
| 6 | Lu-DOTA | — | Arg | Phe | Phe |
| 7 | DOTA | GABA | Arg | Phe | Phe |
| 8 | Ga-DOTA | GABA | Arg | Phe | Phe |
| 9 | DOTA | — | Lys | Phe | Phe |
| 10 | Ga-DOTA | — | Lys | Phe | Phe |
| 11 | DOTA | Gly | Arg | Phe | Phe |
| 12 | DOTA | Ala | Arg | Phe | Phe |
| 13 | Arg-Gly-Asp | — | Arg | Phe | Phe |
| 14 | DOTA | Arg-Gly-Asp | Arg | Phe | Phe |
| 15 | DOTA | 8-aminooctanoic | Arg | Phe | Phe |
| | | acid (ω- | | | |
| | | Aminocaprylic | | | |
| | | acid) C₈H₁₇NO₂ | | | |
| 16 | (DOTA)2 (Bis | Lys | Arg | Phe | Phe |
| | DOTA) | | | | |
| | (C₁₆H₂₈N₄O₈)2 | | | | |
| 17 | DOTA-Lys- | GABA | Lys | Phe | Phe |
| | DPhe-Arg- | | | | |
| | Gly-Asp-Glu | | | | |
| 18 | DOTA | 4-aminomethyl- | Arg | Phe | Phe |
| | | benzoic acid (4- | | | |
| | | Amb) C₈H₉NO₂ | | | |
| 19 | DOTA | Phe-Phe | Arg | Phe | Phe |
| 20 | DOTA | Phe | Arg | Phe | Phe |
| 21 | DOTA | — | Orn | Phe | Phe |
| 22 | DOTA | — | Glu-Glu- | Phe | Phe |
| | | | Orn | | |
| 23 | DOTA | — | Glu-Orn | Phe | Phe |
| 24 | DOTA | — | Lys | Tyr | Phe |
| 25 | DOTA | — | Arg | Tyr | Phe |
| 26 | — | — | Arg | Phe | Tyr |
| 27 | CPT | 8-aminooctanoic | Arg | Phe | Phe |
| | C₂₀H₁₆N₂O₄ | acid | | | |
| | | C₈H₁₇NO₂ | | | |
| 28 | Niraparib | Glutaric acid - | Arg | Phe | Phe |
| | C₁₉H₂₀N₄O | CO(CH₂)₃CO | | | |

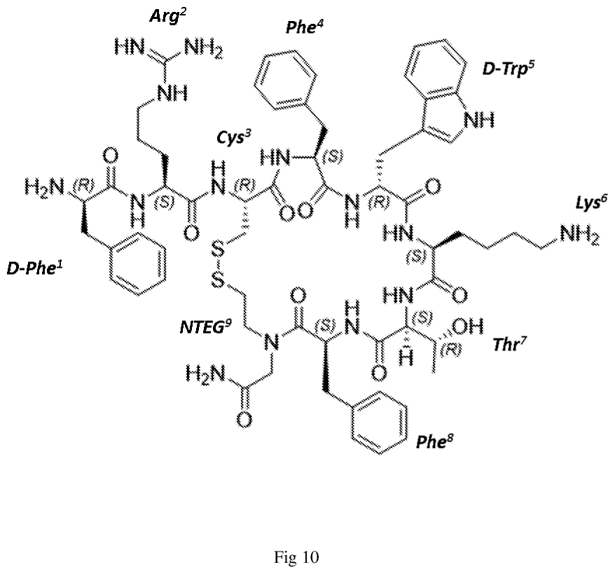

SEQ ID NO: 1 is disclosed in U.S. Pat. No. 10,266,579, having a chemical structure of H-D-Phe¹-Arg²-Cys³-Phe⁴-D-Trp⁵-Lys⁶-Thr⁷-Phe⁸-NTEG⁹-NH² (cyclo 3-9), molecular formula: C₆₁H₈₁N₁₅O₁₀S₂; exact molecular weight: 1248.53 g/mol. The chemical structure of SEQ ID NO: 1 is depicted in FIG. 10. Peptide SEQ ID NO: 1 is a backbone cyclic somatostatin analog. The cyclization between amino acid Cys³ and NTEG⁹ of SEQ ID NO: 1 was seen to constrain the conformation of the corresponding pharmacophore of the native hormone SRIF-14 that is Phe⁶-Phe⁷-D-Trp⁸-Lys⁹-Thr¹⁰-Phe¹¹ by the sequence of Phe¹-Phe⁴-D-Trp⁵-Lys⁶-Thr⁷-Phe⁸. The cyclized pharmacophore is bridged by a disulfide bond. The alkyl side chain of the NTEG⁹ is ethyl (n=2) which is N-thioethyl-glycine or (2-2 -((mercaptoethyl)amino) acetamide) or “GlyS2” building unit.

The in-vitro data disclosed in U.S. Pat. No. 10,266,579 (SEQ ID NO:1 of U.S. Pat. No. 10,266,579) showed that the covalent conjugation of the peptide at the N-terminal to fluorescent marker Fluorescein isothiocyanate (FITC) via the linker Gamma Amino Butyric Acid (GABA) exhibited equal (nonspecific) binding and internalization properties to all somatostatin receptors that are SSTR-1, 2, 3, 4 and 5. It should be emphasized that these analyses were based on the utilization of flow cytometry of Fluorescence-Activated Cell Sorting (FACS) detection of total fluorescence of cellular readout. Therefore, the FACS readouts measured the fluorescence readouts derived from the FITC-GABA-peptides which attached to cellular surface (binding) as well the cellular uptake (internalization). Hence, the FACS indicates the binding of the ligand as an indirect methodology. Herein, SEQ ID NO:1 shows a high affinity and selectivity to human cloned SSTR3 in the nanomolar range, while showing significantly lower affinities to other SSTRs 1, 2, 4 and 5. Surprisingly it exhibits a superagonistic activity profile of SSTR3 above the native hormone SRIF-14 (table 4). Moreover, is exhibits a novel and unexpected in vivo endocrine profile as a potent anti-secretagogue of growth hormone secretion. This indicates for the first time the exact role of SSTR3 in the inhibition of GH release and the potential therapeutic use of SSTR3 selective agonists in diseases and conditions associated with overexpression of SSTR3.

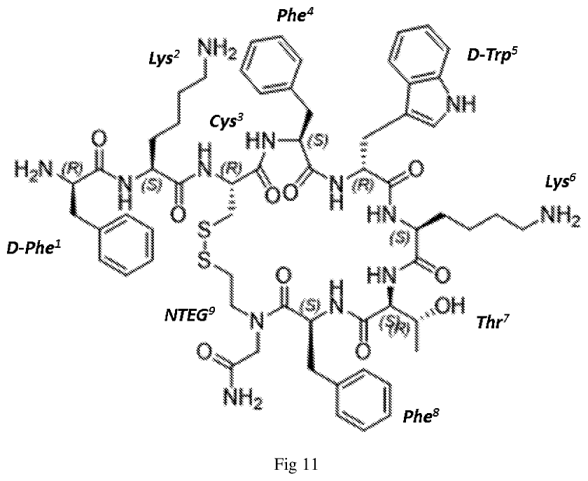

SEQ ID NO: 2 is a novel compound with the chemical formula of H-D-Phe¹-Lys²-Cys³-Phe⁴-D-Trp⁵-Lys⁶-Thr⁷-Phe⁸-NTEG⁹-NH2 (cyclo 3-9). molecular formula: C₆₁H₈₁N₁₃O₁₀S₂; and exact molecular weight: 1220.52 g/mol. The chemical structure of SEQ ID NO: 2 is depicted in FIG. 11.

The SEQ ID NO: 2 is a Lys-2 analog of SEQ ID NO: 1 As described with regard to SEQ ID NO: 1, the position of the amino acid Arg at position 2 has a role of the affinity and selectivity of SEQ ID NO: 1 to SSTR3. However, the physicochemical and pharmaceutical properties of amphiphilic peptides bearing the amino acid Arg might limit their use due to potential surface activity as nonspecific binding and sustained renal clearance. Indeed, in-vivo imaging studies of Positron Emitting Tomography (PET) showed that the systemic administration of the gallium-68 radiolabeled sequences that are analogs of SEQ ID NO: 1 exhibited a sustained renal clearance in mice and rats. When the amino acid Arg was substituted with Lys resulting in SEQ ID NO: 2, surprisingly, it was found that the replacement of Arg with the Lys still retained the binding affinity and selectivity to SSTR3. These results demonstrate that the unique sequence at the N-terminal of Phe-Arg of SEQ ID NO: 1 and the Phe-Lys of the new sequence SEQ ID NO: 2 contribute to the novel specific binding to SSTR3. Emphasizing that both SEQ ID NO: 1 and SEQ ID NO:2 exhibit the same nanomolar affinity to SSTR3 and show the unexpected potent in vivo inhibition of GH release. Further, physicochemical studies showed that SEQ ID NO: 2 had favorable surface activity profile by means of reduced nonspecific binding which may make it a better candidate as radiopharmaceutical ligand to SSTR3.

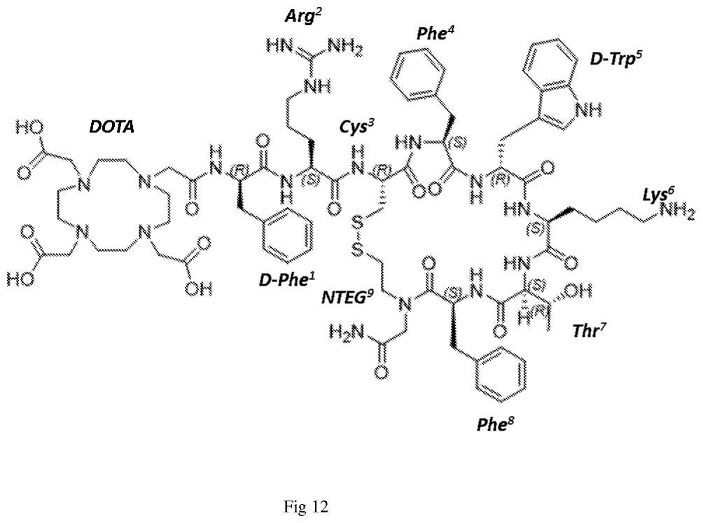

SEQ ID NO: 3 is a novel compound with the chemical formula of DOTA-D-Phe¹-Arg²-Cys³-Phe⁴-D-Trp⁵-Lys⁶-Thr⁷-Phe⁸-NTEG⁹-NH2 cyclo 3-9. Molecular formula: C₇₇H₁₀₇N₁₉O₁₇S₂; and exact molecular weight: 1634.94 g/mol. The chemical structure of SEQ ID NO: 3 is depicted in FIG. 12.

It should be emphasized that DOTA is a bulky moiety with a molecular weight of approximately 400 Daltons and possesses free anionic residues as triactic acids and therefore this conjugation might affect the binding affinity of the sequence. The binding data show that the conjugation of DOTA did not interfere with the binding affinity and selectivity of SEQ ID NO: 1. Therefore, the DOTA conjugate of SEQ ID NO: 1 can be used for radiolabeling with isotopes to enable the diagnosis or treatment of tumors expressing SSTR3 such as functional (GH secreting) pituitary adenoma.

SEQ ID NO: 4 is novel compound with the chemical formula of gallium labeled Ga-DOTA-D-Phe¹-Arg²-Cys³-Phe⁴-D-Trp⁵-Lys⁶-Thr⁷-Phe⁸-NTEG⁹-NH2 (cyclo 3-9). Molecular formula: C₇₇H_{104G}GaN₁₉O₁₇S₂; and exact molecular weight: 1701.64 g/mol.

SEQ ID NO: 4 is an example of R1, where R1 is gallium labeled DOTA conjugated at the N-terminal of SEQ ID NO: 1. It should be emphasized that the chelation of isotope such as gallium by DOTA might affect the binding affinity of the DOTA-peptide conjugate. The binding data show that the gallium labeled DOTA-peptide conjugate did not interfere with the binding affinity and selectivity of SEQ ID NO: 1 and 3. Therefore, the gallium radiolabeled DOTA conjugate of SEQ ID NO: 1 can be used for the diagnosis tumors expressing SSTR3 such as functional (GH secreting) pituitary adenoma. Similar results were found with SEQ ID NO: 5, labeled with copper and SEQ ID NO: 6, labeled with lutetium.

SEQ ID NO: 5 is novel compound with the chemical formula of copper labeled Cu-DOTA-D-Phe¹-Arg²-Cys³-Phe⁴-D-Trp⁵-Lys⁶-Thr⁷-Phe⁸-NTEG⁹-NH2 (cyclo 3-9). Molecular formula: C₇₇H₁₀₅CuN₁₉O₁₇S₂; and exact molecular weight: 1696.47 g/mol.

SEQ ID NO: 6 is novel compound with the chemical formula of lutetium labeled Lu-DOTA-D-Phe¹-Arg²-Cys³-Phe⁴-D-Trp⁵-Lys⁶-Thr⁷-Phe⁸-NTEG⁹-NH2 (cyclo 3-9). Molecular formula: C₇₇H₁₀₄LuN₁₉O₁₇S₂; and exact molecular weight: 1806.88 g/mol.

SEQ ID NO: 7 is novel compound with the chemical formula of DOTA-GABA-D-Phe¹-Arg²-Cys³-Phe⁴-D-Trp⁵-Lys⁶-Thr⁷-Phe⁸-NTEG⁹-NH2 (cyclo 3-9). Molecular formula: C₈₁H₁₁₄N₂₀O₁₈S₂; and exact molecular weight: 1720.05 g/mol.

In SEQ ID NO: 7, R1 is DOTA and R2 is a GABA used as a linker conjugated at the N-terminal of SEQ ID NO: 1. The aim of the GABA as a linker is to increase the distance between the DOTA chelator and peptide sequence. The GABA is used as a spacer and reduces the possible steric effect of the DOTA chelator on the interaction of the peptide ligand with the SSTR3. The binding data show that the conjugation of DOTA via GABA which is covalently bound at the N-terminal of the peptide ligand increased the affinity and selectivity and did not interfere with the binding affinity and selectivity of SEQ ID NO: 1, indicating that SEQ ID NO: 7 may be used for radiolabeling with isotopes to enable the diagnosis or treatment of SSTR3 pituitary adenomas as well as tumors expressing SSTR3.

SEQ ID NO: 8 is novel compound with the chemical formula of gallium labelled DOTA-GABA-D-Phe¹-Arg²-Cys³-Phe⁴-D-Trp⁵-Lys⁶-Thr⁷-Phe⁸-NTEG⁹-NH2 (cyclo 3-9). Molecular formula: C₈H₁₁₁GaN₂₀O₁₈S₂; and exact molecular weight: 1786.74 g/mol.

SEQ ID NO: 8 is an example of isotope labeled of SEQ ID NO: 7. In the case of SEQ ID NO: 8 the labeling with gallium maintains the same affinity and selectivity to SSTR3 as SEQ ID NO. 1, and SEQ ID NO. 3-7. Therefore, the DOTA conjugate of SEQ ID NO: 8 and its isotopes labeled analogs can be used for radiolabeling with isotopes to enable the diagnosis or treatment of SSTR3 pituitary adenomas as well as tumors expressing SSTR3.

SEQ ID NO: 9 is a novel compound with the chemical formula of DOTA-D-Phe¹-Arg²-Cys³-Phe⁴-D-Trp⁵-Lys⁶-Thr⁷-Phe⁸-NTEG⁹-NH2 (cyclo 3-9). Molecular formula: C₇₇H₁₀₇N₁₇O₁₇S₂; and exact molecular weight: 1606.93 g/mol.

In SEQ ID NO: 9, DOTA conjugated at the N-terminal and R3 is Lys. The binding data show that the conjugation of DOTA did not interfere with the binding affinity and selectivity of SEQ ID NO: 2, indicating that the DOTA conjugate of SEQ ID NO: 2 can be used for radiolabeling with isotopes to enable the diagnosis or treatment of tumors expressing SSTR3 such as functional (GH secreting) pituitary adenoma.

SEQ ID NO: 10 is a novel compound with the chemical formula of gallium labeled DOTA-Phe¹-Arg²-Cys³-Phe⁴-D-Trp⁵-Lys⁶-Thr⁷-Phe⁸-NTEG⁹-NH2 (cyclo 3-9). Molecular formula: C₇₇H₁₀₄GaN₁₇O₁₇S₂; and exact molecular weight: 1673.62 g/mol.

In SEQ ID NO: 10 R1 is gallium labeled DOTA conjugated at the N-terminal of SEQ ID NO: 2. As shown below, the binding affinity of the gallium labeled SEQ ID NO: 9 can affect the binding of the ligand to the receptor. The labelling with gallium reduced the IC₅₀ value of 0.73nM (of the parent SEQ ID NO: 2) and approximately 1 nM of SEQ ID NO: 9 to IC₅₀ value of 2.4nM. The binding data show that the affinity of gallium labeled DOTA-peptide conjugate still remained within the nanomolar range of IC₅₀ value of 2.4 nM (which is the same IC₅₀ of the drug octreotide to SSTR2) and selectivity to SSTR3 versus the other SSTRs. Therefore, the gallium radiolabeled DOTA conjugate of SEQ ID NO: 2 can be used for the diagnosis tumors expressing SSTR3.

SEQ ID NO: 11 is a novel compound with the chemical formula of DOTA-Gly¹-D-Phe²-Arg³-Cys⁴-Phe⁵-D-Trp⁶-Lys⁷-Thr⁸-Phe⁹-NTEG¹⁰-NH2 (cyclo 4-10). Molecular formula: C₇₉H₁₁₀N₂₀O₁₈S₂ and exact molecular weight: 1691.99. This sequence is similar to SEQ ID NO: 3, however an amino acid spacer as R2 was introduced.

SEQ ID NO: 12 is a novel compound with the chemical formula of DOTA-Ala¹-D-Phe²-Arg³-Cys⁴-Phe⁵-D-Trp⁶-Lys⁷-Thr⁸-Phe⁹-NTEG¹⁰-NH2 (cyclo 4-10). Molecular formula: C₈₀H₁₁₂N₂O₁₈S₂ and exact molecular weight: 1706.02. This sequence is similar to SEQ ID NO: 3, however an amino acid spacer as R2 was introduced. In this sequence and in SEQ ID NO: 11 similar affinity and selectivity to SSTR3 as SEQ ID NO: 1

SEQ ID NO: 13 is a novel compound with the chemical formula H-Arg¹-Gly²-Asp³-D-Phe⁴-Arg⁵-Cys⁶-Phe⁷-D-Trp⁸-Lys⁹-Thr¹⁰-Phe¹¹ -NTEG¹²-NH² (cyclo 6-12). Molecular formula: C₇₃H₁₀₁N₂₁O₁₅S₂ and exact molecular weight: 1576.86. This sequence is similar to SEQ ID NO: 1, however a tripeptide Arg-Gly-Asp (RGD) which belongs to the family of integrin that is used as a tumor adhesive motif was added to the N-terminus.

SEQ ID NO: 14 is a novel compound with the chemical formula: DOTA-Arg¹-Gly²-Asp³-D-Phe⁴-Arg⁵-Cys⁶-Phe⁷-D-Trp⁸-Lys⁹-Thr¹⁰-Phe¹¹-NTEG¹²- NH2 (cyclo 6-12). Molecular formula: C₈₉H₁₂₇N₂₅O₂₂S₂ and exact molecular weight: 1963.27. This sequence is similar to SEQ ID NO: 13, however a DOTA moiety was added to the N-terminus. In both this sequence and SEQ ID NO: 13, the addition of the RGD moiety did not interfere with the affinity and selectivity of SEQ ID NO: 3 to SSTR3 and therefore can be used to enhance tumor penetration.

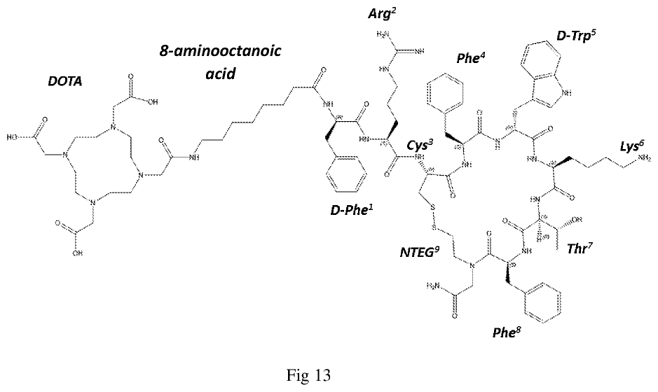

SEQ ID NO: 15 is a novel compound with the chemical formula DOTA-8-aminooctanoic acid-D-Phe¹-Arg²-Cys³-Phe4-D-Trp⁵-Lys⁶-Thr⁷-Phe⁸-NTEG⁹-NH²(cyclo 3-9). Molecular formula: C₈₅H₁₂₂N₂O₁₈S₂ and exact molecular weight: 1776.15. The chemical structure of SEQ ID NO: 15 is depicted in FIG. 13. This sequence is similar to SEQ ID NO: 3, however an 8-aminooctanoic acid spacer as R2 was introduced. This sequence and SEQ ID NO: 11-14 exhibit similar affinity and selectivity to SSTR3 as SEQ ID NO: 1. In addition the 8-aminooctanoic acid linker increases the hydrophobicity of the DOTA-peptide conjugate and therefore can enhance tumor uptake.

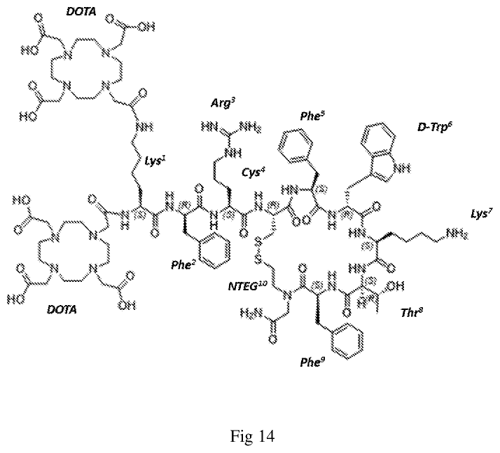

SEQ ID NO: 16 is a novel compound with the chemical formula of (DOTA)2-Lys¹-D-Phe²-Arg³-Cys⁴-Phe⁵-D-Trp⁶-Lys⁷-Thr⁸-Phe⁹-NTEG¹⁰-NH2 (cyclo 3-9). Molecular formula: C₉₉H₁₄₅N₂₅O₂₅S₂; and exact molecular weight: 2149.52. The chemical structure of SEQ ID NO: 16 is depicted in FIG. 14.

The chemical structure of SEQ ID NO: 3 is depicted in FIG. 12. This sequence is similar to SEQ ID NO: 3, however an additional DOTA chelator is conjugated to the sequence via the additional lysine amino acid at the n-terminus. The addition of another DOTA to the SEQ ID NO: 3 did not interfere with the affinity and selectivity to SSTR3 and therefore the additional DOTA enabled an increased specific radioactivity of the tracer. The increased specific radioactivity can enhance the imaging performance of this tracer and the efficacy of targeted radiotherapy of tumors expressing SSTR3 such as functional (GH secreting) pituitary adenoma.

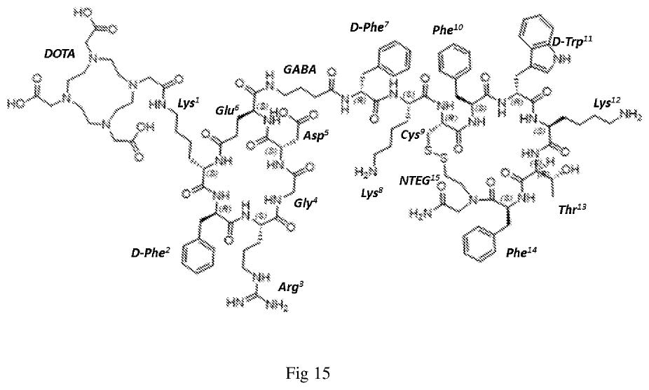

SEQ ID NO: 17 is a novel compound having the structure: DOTA-Lys¹-D-Phe²-Arg³-Gly⁴-Asp⁵-Glu⁶-GABA-D-Phe⁷-Lys⁸-Cys⁹-Phe¹⁰-D-Trp¹¹-Lys¹²- Thr¹³-Phe¹⁴-NTEG¹⁵-NH2 (cyclo 1-6; cyclo 9-15). Molecular formula: C₉₉H₁₃₄N₂₄O₂₀S₂; and exact molecular weight: 2020.41. The chemical structure of SEQ ID NO: 17 is depicted in FIG. 15.

SEQ ID NO: 17 is a bicyclic analog of SEQ ID NO: 2. This sequence has an additional DOTA conjugated cyclic sequence of RGD which linked via GABA at the n-terminal of SEQ ID NO:2. The SEQ ID NO: 17 exhibits similar SSTR3 affinity and selectivity as SEQ ID NO:2 and therefore the additional RGD moiety can be used for enhancing tumor penetration.

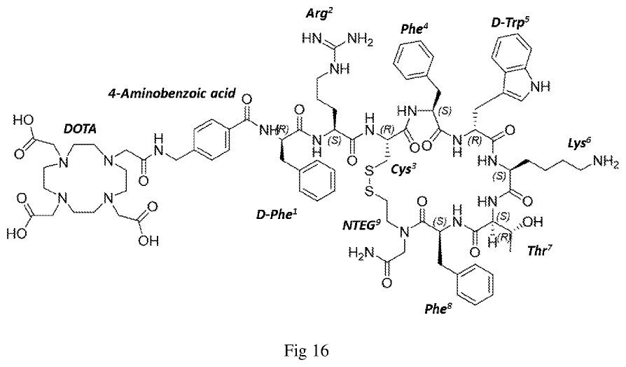

SEQ ID NO: 18 is a novel compound with the chemical formula of DOTA-4-Amb-D-Phe¹-Arg²-Cys³-Phe⁴-D-Trp⁵-Lys⁶-Thr⁷-Phe⁸-NTEG⁹-NH2 (cyclo 3-9). Molecular formula: C₈₅H₁₁₄N₂₀O₁₈S₂; and exact molecular weight: 1768.09. The chemical structure of SEQ ID NO: 18 is depicted in FIG. 16. This sequence is similar to SEQ ID NO: 3, however an aminobenzoic acid (4-Amb) spacer as R2 was introduced. This sequence exhibits similar affinity and selectivity to SSTR3 as SEQ ID NO: 1 and 3. In addition the 4-Amb linker increases the hydrophobicity of the DOTA-peptide conjugate and therefore can enhance tumor uptake.

SEQ ID NO: 19 is a novel compound with the chemical formula DOTA-Phe¹-Phe²-D-Phe³-Arg⁴-(Cys⁵-Phe⁶-D-Trp⁷-Lys⁸-Thr⁹-Phe¹-NTEG¹¹)-NH2 (cyclo 5-11). Molecular formula: CH₁₂₅N₂₁O₁₉S₂; and exact molecular weight: 1929.29. This sequence is similar to SEQ ID NO: 3, however an additional di-Phe sequence was introduced as spacer at R2. This sequence exhibits similar affinity and selectivity to SSTR3 as SEQ ID NO: 3. In addition the di-Phe spacer increases the hydrophobicity of the DOTA-peptide conjugate and therefore can enhance tumor uptake.

SEQ ID NO: 20 is a novel compound with the chemical formula DOTA-Phe¹-D-Phe²-Arg³-(Cys⁴-Phe⁵-D-Trp⁶-Lys⁷-Thr⁸-Phe⁹-NTEG¹⁰)-NH2 (cyclo 4-10). Molecular formula: C₇₀H₉₀N₁₆O₁₁S₂; and exact molecular weight: 1395.71. This sequence is similar to SEQ ID NO: 3, however an additional single Phe was introduced as spacer at R2. This sequence exhibits similar affinity and selectivity to SSTR3 as SEQ ID NO: 3 and 20. In addition the Phe spacer increases the hydrophobicity of the DOTA-peptide conjugate and therefore can enhance tumor uptake.

SEQ ID NO: 21 is a novel compound with the chemical formula of DOTA-D-Phe¹-Orn²-Cys³-Phe⁴-D-Trp⁵-Lys⁶-Thr⁷-Phe⁸-NTEG⁹-NH2 cyclo 3-9. Molecular formula: C₅₀H₇₉N₁₃O₁₀S₂; and exact molecular weight: 1206.49 g/mol. The SEQ ID NO: 21 is a Orn-2 analog of SEQ ID NO: 1 As described with regard to SEQ ID NO: 1, the position of the amino acid Arg at position 2 has a role of the affinity and selectivity of SEQ ID NO: 1 to SSTR3. However, the physicochemical and pharmaceutical properties of amphiphilic peptides bearing the amino acid Arg might limit their use due to potential surface activity as nonspecific binding and sustained renal clearance. The replacement of Arg² with the Orn² did not interfere with the affinity and selectivity to SSTR3. These results demonstrate that the unique sequence at the N-terminal of Phe-Arg of SEQ ID NO: 1, the Phe-Lys of SEQ ID NO: 2 and the Phe-Orn SEQ ID NO: 21 contribute to the novel specific binding and selectivity to SSTR3. The reduction of ion charge from Arg to Lys or Orn can affect the renal clearance and improved the therapeutic profile in tumor imaging and therapy.

SEQ ID NO: 22 is a novel compound with the chemical formula of DOTA-D-Phe¹-Glu²-Glu³-Orn⁴-Cys⁵-Phe⁶-D-Trp⁷-Lys⁸-Thr⁹-Phe¹⁰-NTEG¹¹-NH2 (cyclo 5-11). Molecular formula: C₈₆H₁₁₉N₁₉O₂₃S₂; and Exact molecular weight: 1851.13 g/mol. The SEQ ID NO: 21 is similar to SEQ ID NO: 22 however an additional di-Glu sequence was introduced as R2. As described with regard to SEQ ID NO: 1, the position of the amino acid Arg as a cationic side chain of the amino acid at position 2 has a role of the affinity and selectivity of SEQ ID NO: 1 to SSTR3. However, the physicochemical and pharmaceutical properties of amphiphilic peptides bearing the amino acid Arg might limit their use due to potential surface activity as nonspecific binding and sustained renal clearance. The replacement of Arg² with the Orn² and the addition of two anionic amino acids did not interfere with the affinity and selectivity to SSTR3. These results demonstrate that the unique sequence at the N-terminal of Phe-Arg of SEQ ID NO: 1, the Phe-Lys of SEQ ID NO: 2, the Phe-Orn SEQ ID NO: 21 and the Phe-Glu-Glu-Orn of SEQ ID NO: 22 contribute to the novel specific binding and selectivity of these DOTA-peptides to SSTR3. The reduction of ion charge from Arg to Lys or Orn and the additional opposite charge of anionic moieties increased the hydrophilicity and can improve the renal clearance and therapeutic profile in tumor imaging and therapy.

SEQ ID NO: 23 is a novel compound with the chemical formula of DOTA-D-Phe¹-Glu²-Orn³-Cys⁴-Phe⁵-D-Trp⁶-Lys⁷-Thr⁸-Phe⁹-NTEG¹⁰-NH2 (cyclo 4-10). Molecular formula: C₈₆H₁₁₉N₁₉O₂₃S₂; and exact molecular weight: 1722.01 g/mol. This sequence is similar to SEQ ID NO: 22, however an additional single Glu (instead of di-Glu) was introduced as spacer at R2. This sequence exhibits similar affinity and selectivity to SSTR3 as SEQ ID NO: 22. The reduction of di-Glu to single Glu can improve the renal clearance versus tumor uptake.

SEQ ID NO: 24 is a novel compound with the chemical formula of DOTA-D-Phe¹-Lys²-Cys³-Tyr⁴-D-Trp⁵-Lys⁶-Thr⁷-Phe⁸-NTEG⁹ -NH2 (cyclo 3-9). Molecular formula: C₆₁H₈₁N₁₃O₁₁O₂; and Exact molecular weight: 1236.52 g/mol. This sequence is similar to SEQ ID NO: 2 and 9, however the Phe at position 4 was replaced by Tyr as R4. This sequence exhibits similar affinity and selectivity to SSTR3 as SEQ ID NO: 2. The replacement of Phe⁴ with Tyr⁴ enhances the hydrophilicity of the DOTA-peptide conjugate and therefore can improve the renal clearance and therapeutic index of the radiolabeled tracer. It can be used, also, as an additional site of radiolabeling of the peptide for example, but not limited to, ¹²⁵I. The additional site of radiolabeling (DOTA and Tyr) will increase the specific radioactivity of the tracer and will enable better imaging performance and radioactive treatment of tumors.

SEQ ID NO: 25 is a novel compound with the chemical formula of DOTA-D-Phe¹-Arg²-Cys³-Tyr⁴-D-Trp⁵-Lys⁶-Thr⁷-Phe⁸-NTEG⁹-NH2 (cyclo 3-9). Molecular formula: C₆₁H₈₁N₁₅O₁₁S₂; and exact molecular weight: 1264.53 g/mol. This sequence is similar to SEQ ID NO: 1 and 3, however the Phe at position 4 was replaced by Tyr as R4. This sequence exhibits similar affinity and selectivity to SSTR3 as SEQ ID NO: 1. The replacement of Phe⁴ with Tyr⁴ enhances the hydrophilicity of the DOTA-peptide conjugate and therefore can improve the renal clearance and therapeutic index of the radiolabeled tracer. It can be used, also, as additional site of radiolabeling of the peptide for example, but not limited to, ¹²⁵I. The additional site of radiolabeling (DOTA and Tyr) will increase the specific radioactivity of the tracer and will enable better imaging performance and radioactive treatment of tumors.

SEQ ID NO: 26 is a novel compound with the chemical formula of H-Phe¹-Arg²-Cys³-Phe⁴-D-Trp⁵-Lys⁶-Thr⁷-Tyr⁸-NTEG⁹-NH2 (cyclo 3-9). Molecular formula: C₆₁H₈₁N₁₅O₁₁S₂; and exact molecular weight: 1264.53 g/mol. This sequence is similar to SEQ ID NO: 1 and 3, however the Phe at position 8 was replaced by Tyr. This sequence exhibits similar affinity and selectivity to SSTR3 as SEQ ID NO: 1. The replacement of Phe⁸ with Tyr⁸ enhances the hydrophilicity of the DOTA-peptide conjugate and therefore can improve the renal clearance and therapeutic index of the radiolabeled tracer. It can be used, also, as additional site of radiolabeling of the peptide for example, but not limited to, ¹²⁵I. The additional site of radiolabeling (DOTA and Tyr) will increase the specific radioactivity of the tracer and will enable better imaging performance and radioactive treatment of tumors.

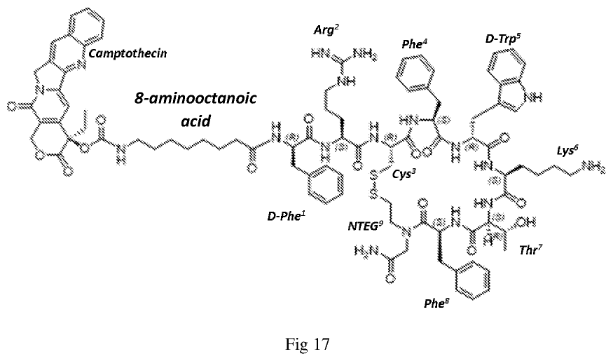

SEQ ID NO: 27 is a novel compound with the chemical formula: CPT-8-aminooctanoic acid-D-Phe¹-Arg²-Cys³-Phe⁴-D-Trp⁵-Lys⁶-Thr⁷-Phe⁸-NTEG⁹-NH2 (cyclo 3-9). Molecular formula: C₉₀H₁₁₀N₁₈O₁₆S₂; exact molecular weight: 1764.1. The chemical structure of SEQ ID NO: 27 is depicted in FIG. 17. This sequence is similar to SEQ ID NO: 1, however the camptothecin (CPT), a cytotoxic and bulky moiety as R1, is covalently bound to the SEQ ID NO: 1 via a linker which is a spacer of 8-aminooctanoic acid, as R2, at the N-terminal of the peptide. SEQ ID NO: 27 exhibits the same selectivity to SSTR3 as SEQ ID NO: 1. The affinity to SSTR3 was reduced but remained within the 10 nM range. The use of the spacer in this sequence reduced the steric effect of the CPT by increasing the distance between the CPT and the pharmacophore of SEQ ID NO: 1. This analog can be used for the delivery and targeting of tumors of the cytotoxic payload.

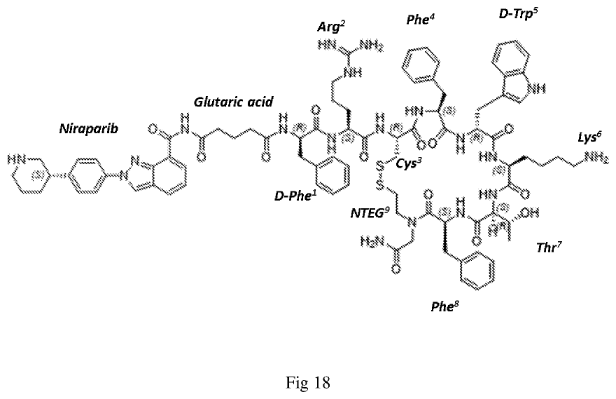

SEQ ID NO: 28 is a novel compound with the chemical formula: Niraparib-CO(CH₂)₃CO-D-Phe¹-Arg²-Cys³-Phe⁴-D-Trp⁵-Lys⁶-Thr⁷-Phe⁸-NTEG⁹-NH2 cyclo 3-9, Molecular formula: C₈₅H₁₀₅N₁₉O₁₃S₂; Exact molecular weight: 1665.02. The chemical structure of SEQ ID NO: 28 is depicted in FIG. 18.

This sequence is similar to SEQ ID NO: 1, however the anticancer PARP inhibitor, a cytotoxic and bulky moiety as R1, is covalently bound to the SEQ ID NO: 1 via a linker which is a spacer of glutaric acid, as R2, at the n-terminal of the peptide. The SEQ ID NO: 28 exhibit the same selectivity to SSTR3 as SEQ ID NO: 1. The affinity to SSTR3 was reduced but remained within the 10nM range. The use of the spacer in this sequence reduced the steric effect of the CPT by increasing the distance between the niraparib and the pharmacophore of SEQ ID NO: 1. This analog can be used for the delivery and targeting of tumors of the cytotoxic payload.

### Somatostatin Analogs

The substantial evidence of overexpressed somatostatin receptors in GEP-NETs was the key rationale for the developments and approval of several radiolabeled somatostatin analogs for the diagnosis and treatment (theragnostics) of GEP-NETs.

The development of these radioligands was based on the pharmacophore (minimal amino acids sequence which exhibits bioactivity) of the drug Octreotide. They all share the specific displacement of Phe amino acid at position 3 of Octreotide with Tyr and they differ from each other by the threonine c-terminal as alcohol (Threoninol as for Octreotide or TOC) or as carboxylic c-terminal (Threonine as for Octreotate TATE). This specific displacement enhanced their high affinity and specificity to SSTR2. Their conjugation at the N-terminus to the metal chelator DOTA (1,4,7,10-Tetraazacyclododecane-1,4,7,10-tetraacetic acid) enabled their radiolabeling with various radioisotopes such as the approved Gallium-68 or Lutetium-177 radioligands. The radiolabeling with Gallium-68 enabled the developments and approval of 68Ga-DOTA-TOC (SomaKit®) and 68Ga-DOTA-TATE (NetSpot®) for the diagnosis of GEP-NET by Positron Emission Tomography (PET). The successful implementation of radiolabeling via the DOTA chelator for the diagnosis of GEP-NET enabled the development and approval of the first peptide receptor radioactive therapy (PRRT) with Lutetium-177. This radiolabeled peptide-based drug is 177Lu-DOTA-TATE and is known as Lutathera® which approved for the treatment of GEP-NET. The rationale and mechanism of drug action of Lutathera® ascribed to the observed internalization of the receptor—peptide complex by endocytosis following the binding of the ligand to the somatostatin receptor. This internalization enabled the radiopeptide to be retained in the receptor-expressing tumor cells, and due to its relatively low molecular weight, and it is rapidly cleared from blood.

### Therapeutic Applications

It is noted that the main limitation associated with the clinical use of the available somatostatin radioligands is their specificity to SSTR-2 alone. There are tumors that express receptors other than SSTR-2 and the expression pattern of SSTR-2 can be changed via the course of the tumor growth versus dynamic overexpression of other somatostatin receptors such as SSTR-3. Unlike SSTR-2 which is expressed in many organs in both normal and disease conditions, SSTR-3 is overexpressed in the normal brain, pituitary, vascular system, thymus, and testis. However, SSTR-3 becomes overexpressed in disease conditions such as, cancer. Moreover, the SSTR3 is the only somatostatin receptor which linked to the activation of signal transduction via p53 induced apoptosis and cell cycle arrest, and the only receptor reported to induce significant higher internalization among the other somatostatin receptors. These differentiated profiles make SSTR-3 as a potential diagnostic and therapeutic target in cancer. More specifically, based on the unexpected in vivo results of both SEQ ID NO: 1 and SEQ ID NO: 2 of inhibition of GH release, these two sequences and their analogs may be drug candidates for the diagnosis and treatment of acromegaly. Acromegaly is caused by a noncancerous (benign) tumor (adenoma) of the pituitary gland. The tumor produces excessive amounts of growth hormone, causing many of the signs and symptoms of acromegaly. Therefore, the SSTR3 specific SEQ ID NO:1 and SEQ ID NO:2 and their analogs can be used for either inhibition of growth hormone release as free (not conjugated) peptide sequence or as conjugated peptides, for example to DOTA for the radioactive diagnosis such as gallium or copper labeled DOTA chelator or for therapy by lutetium or actinium labeled DOTA.

Disclosed herein is the synthesis and identification of a novel somatostatin analogs which possesses high affinity and specificity to SSTR-3. In-vitro binding and bioactivity studies showed that these novel SSTR-3 act as super-agonist and induced significant internalization of the SSTR-3 into cells. Therefore, these novel somatostatin analogs can be used as a radioligand (conjugated to various radioisotopes and chelator/s) for the use as a theragnostic entity. These potential applications may include their uses for the diagnosis and PRRT of tumors that overexpress specifically SSTR3. The expression of SSTR-3 was indicated in various malignancies that include GEP-NET and other tumors such as pituitary adenomas, blood malignancies that include, but are not limited to, sarcoma, myeloma, lymphoma and leukemia, and solid tumors that include, but are not limited to, pancreatic, colon, breast, prostate, ovarian, liver, kidneys and lungs (unpublished empirical data of SSTR3 expression in human tumors, data is not shown).

It should be emphasized that the potential uses of the disclosed novel SSTR-3 ligands as a theragnostic radioligand, can be used for the diagnosis and treatment of various anomalies where SSTR-2 or the non-selective analogs have limited efficacy.

Another potential indication for the described novel SSTR-3 analogs is their potential use in cancer treatment. The specific signal transduction of SSTR-3 and apoptosis may enable the use of these ligands as either monotherapy or synergists of cytotoxic chemotherapies and radiation of various tumors. The activation of SSTR-3 by the described novel super-agonists may increase the sensitivity of cancer cells to the clinical available cytotoxic remedies.

Another indication of the described novel SSTR-3 analogs is the indicated role of SSTR-3 in angiogenesis of tumors. The interaction and activation of SSTR-3 by super agonists may result with antiangiogenic effect which may lead to tumor suppression or remission.

Further indications of the described novel SSTR-3 analogs include their therapeutic potential of inhibitor of growth hormone and IGF-1 release and synthesis. This endocrine effect may reduce the growth hormone levels in acromegaly and in other anomalies associated with the activation of GH-IGF-1 axis, that include, but not limited to, type (II) diabetes, diabetic nephropathy and retinopathy and dawn syndrome.

Another indication of the described novel SSTR-3 analogs is their therapeutic potential in Cushing disease. The indicated expression of SSTR-3 in the corticoadrenal may indicated on the role of somatostatin as inhibitor of cortisol release as well as the inhibition of ACTH release from the pituitary.

Additional indications of the described novel SSTR-3 analogs include their therapeutic potential in inflammation and immune diseases. The indicated expression of SSTR-3 in human immune cells such as B and T-lymphocytes, monocytes and human thymus may be used as inhibitor of vascular permeability and to reduce inflammation by inhibiting lymphocytes proliferation and the secretion of proinflammatory cytokines. The overexpression of SSTR-3 in the thymus may be used as activation of the SSTR-3 for immunosuppression via inhibition of production of B lymphocytes.

Another indication of the described novel SSTR-3 analogs is their therapeutic potential in male spermatogenesis. The overexpression of SSTR-3 in testis may indicated the therapeutic potential of SSTR-3 activation in various anomalies associated with male fertility.

As demonstrated in the examples of the present application, SEQ ID NO: 1 and SEQ ID NO: 2 and their analogs displayed high binding affinity and selectivity to SSTR-3. This data indicates that these sequences can be used as drugs for the treatment of oversecreting functional pituitary adenomas associated with deregulated growth hormone release. For example, SEQ ID NO: 1 and SEQ ID NO:2 and their analogs may be beneficial to selectively reduce the release of growth hormone (from the pituitary) of acromegaly patients without the adverse inhibition of pancreatic hormones of insulin and glucagon associated with SSTR-2, as occurs in commonly used drugs such as Octreotide, Lanreotide and Pasireotide.

A chelator-based derivative of SEQ ID NO: 1 and SEQ ID NO:2 and their analogs, wherein the chelator may be DOTA, is a possible embodiment of the claimed invention. The radiopharmaceutical remedy of DOTA conjugated to SEQ ID NO: 1 and SEQ ID NO: 2 and their analogs can be used for the diagnosis and treatment of cancer patients who have tumors with overexpression of SSTR-3. For use of diagnosis of cancer, the conjugated DOTA to SEQ ID NO: 1 and SEQ ID NO: 2 and their analogs radiolabeled with positron emitting radioisotopes such as, but not limited to, Copper-64 or Gallium-68. For the use of cancer treatment, the conjugated DOTA to SEQ ID NO: 1 and SEQ ID NO: 2 and their analogs radiolabeled with positron emitting radioisotopes such as Lutetium-177, Indium-111 or Actinum-225.

In certain embodiments the descried sequences are used in the treatment of acromegaly, Cushing syndrome, Dawn Syndrome, Nephropathy, Retinopathy, inflammation, immune disorders and cellular senescence-related male infertility.

In certain embodiments the descried sequences are used in the treatment of cancer. In some examples cancer is a hematological tumor including leukemias, acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. In some embodiments the cancer is a solid tumor, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers (such as small cell lung carcinoma and non-small cell lung carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma, astrocytoma, medulloblastoma, craniopharyogioma, ependymoma, pinealoma, hemangioblastoma, acoustic neuroma, oligodendroglioma, menangioma, neuroblastoma and retinoblastoma). In some embodiments the cancer is a neuroendocrine cancer, such as Adrenal cancer, Carcinoid tumors, Merkel cell carcinoma, Pancreatic neuroendocrine tumors, Paraganglioma, Pheochromocytoma, Medullary thyroid carcinoma, Pheochromocytoma of the adrenal gland, Small cell carcinoma, and Large cell carcinoid tumor. In some embodiments the cancer is a vascular tumor, such as vascular tumors such as angiosarcoma, infantile hemangioma, congenital hemangioma, kaposiform hemangioendothelioma (KHE), and pyogenic granuloma.

For a diagnostic use of one of the sequences described herein, a preferred dose is an amount of between 40 micrograms (μg) and 200 μg per 60 kg body weight. For a therapeutic use of one of the sequences described herein, a preferred dose is an amount of between 200 micrograms (μg) and 800 μg per 60 kg body weight.

Embodiments described herein relate to: A somatostatin analog having the formula: R¹-R²-DPhe-R³-Cys-R⁴-DTrp-Lys-Thr-R⁶-R⁵ wherein R¹ is an active agent, or is absent; R² is a linker, an active agent, or is absent; R³ is either Arg, Lys, or Orn; or optionally a polypeptide of 3 or two amino acids Glu-Glu-R⁷ or Glu-R⁷, wherein R⁷ is Arg, Lys, or Orn; R⁴ is either Phe or Tyr; and; R⁵ is a NTAG having as structure of N-ThioAlkyl-Glycine

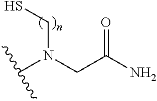 [SMILES: *N(CS)CC(N)=O]

wherein optionally a disulfide bond is formed between R⁵ and the cysteine residue or a pharmaceutically acceptable salt thereof, and n is the number of methylene groups from 1 to 5; R⁶ is either Phe or Tyr; wherein when R³ is Arg, either R⁴ or R⁶ is Tyr. Optionally, R³ is Lys. Optionally, R⁴ and R⁶ are Phe. Optionally, R³ is Arg and either R⁴ or R⁶ is Tyr. Optionally, R¹ comprises one or more active agents. Optionally, the active agent is selected from the group consisting of: an imaging moiety, a therapeutic moiety, a dye, a fluorescent moiety, a toxin, a chelator, a metal atom moiety, a radioactive atom moiety, a nanoparticle, an ethylene glycol polymer, a photosensitizer, a liposome constituent micelle constituent and a tumor targeting moiety, such as a lipid or RGD. Optionally, R¹ is a chelator moiety. Optionally, R² is a linker selected from the group consisting of: gamma-aminobutyric acid, between 1 and 3 amino acids, aminooctanoic acid, 4-aminomethyl-benzoic acid, and glutaric acid. Optionally, R¹ or R² comprises the amino acid sequence, Arg-Gly-Asp. Optionally, the active agent moiety is a chelator moiety, selected from the group consisting of: DOTA (1,4,7,10-tetraazacycl ododecane-1,4,7,10-tetraacetic acid), NOTA (2-(4,7-bis(2-(tert-butoxy)-2-oxoethyl)-1,4,7-triazonan-1-yl) acetic acid), NODA (4-(4,7-bis(2-(tert-butoxy)-2-oxoethyl)-1,4,7-triazacyclononan-1-yl)-5 -(tert-butoxy)-5-oxopentanoic acid) and EDTA (ethylenediaminetetraacetic acid). Optionally, the active agent moiety comprises a metal atom selected from the group consisting of gallium, copper, and lutetium. Optionally, the active agent moiety is a radioactive atom-comprising moiety, comprising an atom selected from the group consisting of: iodine-123, iodine-125, iodine-131, fluorine-18, carbon-11, carbon-14, tritium, nitrogen-13, oxygen-15 and phosphorous-32, technetium-99m, chromium-51, cobalt-57, cobalt-58, erbium-169, gallium-67, gallium-68, copper-64, indium-111, iron-59, lutetium-175, lutetium-177, radium-223, rubidium-82, samarium-153, selenium-75, strontium-89, thallium-201 and yttrium-90. Actinium-225. Optionally, the active agent moiety is a photosensitizer selected from the group consisting of a phenothiazine, a xanthene and a porphyrin. Optionally, the active agent moiety is a toxin selected from the group consisting of niraparib, actinomycin, camptothecin, doxorubicin, and gentamicin. Optionally, R³ is Lys, or Orn. Optionally, R⁴ is Phe. Optionally, n is 2. Optionally, the somatostatin analog according to has the structure of SEQ ID NO: 2, 9, 10, 17, 21, 22, 23 or 24. Further described is a pharmaceutical composition comprising at least one somatostatin analog, and at least one pharmaceutically acceptable excipient. Optionally, the somatostatin analog or pharmaceutical composition is for use in treatment or diagnosis of a disease associated with SSTR3. Optionally, the disease is selected from the group consisting of: cancer, a tumor, acromegaly, type (II) diabetes, diabetic nephropathy, diabetic retinopathy or dawn syndrome, Cushing disease, inflammation, immune disorders, cellular senescence, and male infertility. somatostatin analog the cancer is selected from the group consisting of: leukemia, acute leukemia, lymphocytic leukemia, acute myelocytic leukemia, acute myelogenous leukemia and myeloblastic, promyelocytic, myelomonocytic, monocytic and erythroleukemia, chronic leukemias, myelocytic (granulocytic) leukemia, chronic myelogenous leukemia, chronic lymphocytic leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. a solid tumor, sarcomas, carcinomas, fibrosarcoma, myxosarcoma, liposarcoma, chondrosarcoma, osteogenic sarcoma, synovioma, mesothelioma, Ewing's tumor, leiomyosarcoma, rhabdomyosarcoma, colon carcinoma, lymphoid malignancy, pancreatic cancer, breast cancer, lung cancers (such as small cell lung carcinoma and non-small cell lung carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, CNS tumors, glioma, astrocytoma, medulloblastoma, craniopharyogioma, ependymoma, pinealoma, hemangioblastoma, acoustic neuroma, oligodendroglioma, menangioma, neuroblastoma, retinoblastoma, neuroendocrine cancer, adrenal cancer, carcinoid tumors, Merkel cell carcinoma, pancreatic neuroendocrine tumors, paraganglioma, pheochromocytoma, medullary thyroid carcinoma, pheochromocytoma of the adrenal gland, small cell carcinoma, large cell carcinoid tumor, a vascular tumor, angiosarcoma, infantile hemangioma, congenital hemangioma, kaposiform hemangioendothelioma (KHE), and pyogenic granuloma.

According to an embodiment, described herein is a somatostatin analog for use in treatment or diagnosis of a disease associated with growth hormone release, or of a disease associated with cancer associated with overexpression of SSTR3; the analog having the formula: R¹-R²-DPhe-R³-Cys-R⁴-DTrp-Lys-Thr-R⁶-R⁵ wherein R¹ is an active agent, or is absent; R² is a linker, an active agent, or is absent; R³ is either Arg, Lys, or Orn; or optionally a polypeptide of 3 or two amino acids Glu-Glu-R⁷ or Glu-R⁷, wherein R⁷ is Arg, Lys, or Orn; R⁴ is either Phe or Tyr; and R⁵ is a NTAG having as structure of N-ThioAlkyl-Glycine

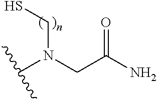 [SMILES: *N(CS)CC(N)=O]

and n is the number of methylene groups from 1 to 5; and R⁶ is either Phe or Tyr, wherein optionally a disulfide bond is formed between R⁵ and the cysteine residue; or a pharmaceutically acceptable salt thereof. Optionally, R¹ comprises one or more active agents. Optionally, wherein the active agent is selected from the group consisting of: an imaging moiety, a therapeutic moiety, a dye, a fluorescent moiety, a toxin, a chelator, a metal atom moiety, a radioactive atom moiety, a nanoparticle, an ethylene glycol polymer, a photosensitizer, a liposome constituent micelle constituent and a tumor targeting moiety, such as a lipid or RGD. Optionally, R¹ is a chelator moiety. Optionally, R² is a linker selected from the group consisting of: gamma-aminobutyric acid, between 1 and 3 amino acids, aminooctanoic acid, 4-aminomethyl-benzoic acid, and glutaric acid. Optionally, R¹ or R² comprises the amino acid sequence, Arg-Gly-Asp. Optionally, the active agent moiety is a chelator moiety, selected from the group consisting of: DOTA (1,4,7,10-tetraazacycl ododecane-1,4,7,10-tetraacetic acid), NOTA (2-(4,7-bis(2-(tert-butoxy)-2-oxoethyl)-1,4,7-triazonan-1-yl) acetic acid), NODA (4-(4,7-bis(2-(tert-butoxy)-2-oxoethyl)-1,4,7-triazacyclononan-1-yl)-5-(tert-butoxy)-5-oxopentanoic acid) and EDTA (ethylenediaminetetraacetic acid). Optionally, the active agent moiety comprises a metal atom selected from the group consisting of gallium, copper, and lutetium. Optionally, the active agent moiety is a radioactive atom-comprising moiety, comprising an atom selected from the group consisting of: iodine-123, iodine-125, iodine-131, fluorine-18, carbon-11, carbon-14, tritium, nitrogen-13, oxygen-15 and phosphorous-32, technetium-99m, chromium-51, cobalt-57, cobalt-58, erbium-169, gallium-67, gallium-68, copper-64, indium-111, iron-59, lutetium-175, lutetium-177, radium-223, rubidium-82, samarium-153, selenium-75, strontium-89, thallium-201 and yttrium-90. Actinium-225. Optionally, the active agent moiety is a photosensitizer selected from the group consisting of a phenothiazine, a xanthene and a porphyrin. Optionally, the active agent moiety is a toxin selected from the group consisting of niraparib, actinomycin, camptothecin, doxorubicin, and gentamicin. Optionally, R³ is either Arg, Lys, or Orn. Optionally, R⁴ is Phe. Optionally, R⁶ is Phe. Optionally, n is 2. Optionally, the disease associated with growth hormone release is acromegaly, type (II) diabetes, diabetic nephropathy, diabetic retinopathy or dawn syndrome. Optionally, the somatostatin analog for use has the structure of any one of SEQ ID NOs 1-28. Optionally, the somatostatin analog is administered in an amount of between 40 and 800 micrograms per administration.

The following examples are provided to illustrate certain particular features and/or embodiments. These examples should not be construed to limit the disclosure to the particular features or embodiments described.

### EXAMPLES

### Example 1: Binding Profile of Somatostatin Analogs to Human Recombinant Somatostatin Receptors

In this example the affinity of the test compound(s) to human cloned somatostatin receptors 1, 2, 3, 4, and 5 was evaluated.

Reference standards were run as an integral part of each assay to ensure the validity of the results obtained. The somatostatin analogs were tested for their affinity and potency by measurements of their potency in inhibition of the binding of ¹²⁵I-Tyr¹¹-SRIF-14 to membrane preparations of transfected cells expressing the transmembrane somatostatin receptors. The binding to human cloned receptors SSTR-1, or SSTR-2 or SSTR-3 evaluated in stable and selective transfected CHO (Chinese Hamster Ovary) cells and binding to STSR-4 and SSTR-5 in stable and selective transfected Chem-1 (Rat ChemiSCREEN) cells. Cell membranes were homogenized in Tris buffer in the presence of protease inhibitors and incubated for 2 hours (SSTR-1, 3,4,5) and 4 hours (SSTR-2) with the radiolabeled ligand ¹²⁵I-Tyr¹¹-SRIF-14 with different concentrations of the tested peptide. The binding reactions were filtered, the filters were washed, and the bound radioactivity was counted using a gamma counter. Nonspecific binding was defined in the presence of 1.0 μM unlabeled SRIF-14.

### Solid Phase Peptide Synthesis (SPPS) of the Backbone Cyclic Somatostatin Receptor 3 Peptides

Synthesis of SEQ ID NO: 1—Rink Amide MBHA resin (100-200 mesh, 1% DVB, 0.77 meq/g, Lot: #3228 “Chem-Impex”). Rink amide MBHA resin (1.5 g, total scale 1.155 mmol) was pre-swollen in DMF overnight. While shaking in a reaction vessel equipped with sintered glass bottom. The Fmoc protecting group was removed from the resin by reaction with 20% piperidine in DMF (15 mL, 2×15 min) followed by DMF wash (15 mL×7×2 min). Fmoc removal was monitored by ninhydrin test (positive). A coupling cycle was carried out with Fmoc-NTEG(Acm)-OH building unit (1.484 g, 3.465 mmol, 3 eq), HOBt monohydrate (531 mg, 3.465 mmol), DIC (536.5 mL, 3.465 mmol) in DMF (16 mL) for 2 h at room temperature. Reaction completion was monitored using qualitative ninhydrin test (Kaiser test, negative). Following coupling, the peptidyl resin was washed with DMF (15 mL, 7×1 min). Fmoc removal of the building unit and washing steps were carried out as described above. Coupling steps of Fmoc-Phe-OH, Fmoc-Thr(t-Bu)-OH, Fmoc-Lys(Boc)-OH, Fmoc-D-Trp(Boc)-OH, Fmoc-Phe-OH, Fmoc-Cys(Acm)-OH, Fmoc-Arg(Pbf)-OH were carried out as described above (Fmoc-AA-OH 3.465 mmol, HOBt monohydrate 3.465 mmol, DIC 3.465 mmol in 16 ml DMF, 1.5 h at rt), monitored by ninhydrin test. Coupling of Fmoc-Arg(Pbf)-OH was repeated by addition of a mixture of Fmoc-Arg(Pbf)-OH (2.248 g, 3.465 mmol), PyBroP (1.615 g, 3.465 mmol), DIEA (1.21 mL, 6.93 mmol) in 15 mL DMF, reaction time over night at room temperature. Coupling of Fmoc-D-Phe-OH was carried out with PyBroP under the same reaction conditions as describe for Fmoc-Arg(Pbf)-OH, reaction time 1.5 h at room temperature (rt).

Cyclization (S-S bridge formation): The peptidyl-resin was washed with DMF/H₂O 4:1 (15 mL×2×2 min) followed by addition of 12 (2.93 g, 11.55 mmol, 10 eq) in DMF/H₂O 4:1 (30 mL), 40 min at rt. The peptidyl-resin was washed (DMF/H₂O 4:1, 8×1.5 min, DMF 5×1.5 min, DCM 3×1.5 min, CHCl₃ 3×1.5 min, 2% ascorbic acid in DMF 5×2 min, DMF 5×1.5 min).

After cyclization, the resin was split into two fractions in 4:1 ratio. Final Fmoc removal of ⅕ of the peptidyl-resin (0.231 mmol) was carried out with 20% piperidine in DMF (15 mL, 2×15 min) followed by DMF wash (7×2 min). Following Fmoc removal, the peptidyl-resin was washed with DCM (5×1 min) and dried under reduced pressure. The peptide was cleaved from the resin by reaction with 7 mL cold mixture of 94% TFA, 3% H₂O and 3% TIS at 0° C. for 15 min and 1.5 h at rt. The resin was removed by filtration. The filtrate was evaporated under nitrogen and the oily product obtained was triturated with cold diethyl ether followed by decantation of the ether fraction. The precipitation was washed with cold diethyl ether for several times to give white powder which was dried under reduced pressure (182 mg of crude peptide, LC-MS: YS-001-010-B).

Synthesis of SEQ ID NO: 2—was synthesized according to the above method, with the substitutions at positions of each sequence as depicted in the detailed description above.

Synthesis of sequences in which R¹ is DOTA: Final Fmoc-deprotection of the protected peptidyl-resin (0.154 mmol) was carried out using 20% piperidine in DMF (2.5 mL, 2×15 min) followed by DMF wash (2.5 mL, 7×2 min). Following Fmoc-deprotection, coupling of DOTA(OtBu)₃ to the peptidyl resin (0.154 mmol) was performed twice using DOTA(OtBu)₃ (265 mg, 0.462 mmol), PyBroP (215 mg, 0.462 mmol), DIEA (161 μL, 0.924 mmol) in DMF (2 mL). The mixture was stirred overnight at rt. Following coupling, the resin was washed with DMF (2.5 mL, 5×1.5 min).

Synthesis of sequences in which R¹ is CPT: DIEA (724 μL, 4.16 mmol) was added to a suspension of premade 4-nitrophenylcarbonate derivative of CPT (1.067 g, 2.079 mmol) in DMF (25 mL). The suspension was stirred for 30 min. Catalytic amount of DMAP in DMF (1 mL) was added to the suspension. The suspension was added to the exposed primary amine of the protected peptidyl-resin (0.693 mmol). The mixture was stirred overnight at rt. Following coupling, the resin was washed with DMF (10 mL, 5×1.5 min).

Synthesis of sequences in which R¹ is Niraparib: Final Fmoc-deprotection of the protected peptidyl-resin (0.0624 mmol) was carried out with 20% piperidine in DMF (1 mL, 2×15 min) followed by DMF wash (1 mL, 7×2 min). The peptidyl-resin was reacted with glutaric anhydride (107 mg, 0.936 mmol), DIEA (326 μL, 1.872 mmol) in DMF (3.5 mL) for 40 min at rt. The resin was washed with DMF (1 mL, 5×1.5 min). The preactivation of the carboxylic moiety was performed using PyBrop (291 mg, 0.624 mmol), DIEA (217 μL, 1.248 mmol) in DMF (2.7 mL) for 20 min at rt. The resin was washed with DMF (1 mL, 5×1.5 min). Coupling of Niraparib to the peptidyl resin (0.0624 mmol) was performed using Niraparib (50 mg, 0.156 mmol), DIEA (65 μL, 0.3744 mmol), catalytic amount of DMAP in DMF (1 mL). The mixture was stirred for 3 h at 50° C. Following coupling, the resin was washed with DMF (1 mL, 5×1.5 min).

The additional sequences were synthesized using the same procedures described above, while making appropriate adaptations for R¹ and R² according to the sequence.

The following table depicts the conditions of binding assay to each of the somatostatin receptors used for the evaluations of binding affinities of the tested peptides. All assays were performed by quantitation method of radioligand binding under 25° C. The vehicle was 1% DMSO, the incubation buffer was 25 mM HEPES, pH 7.4, 5 mM MgCl₂, 1 mM CaCl₂, 0.1% BSA. The non-specific binding ligand was 1.0 μM SRIF-14. The significance criteria were >50% of max inhibition. Table 2 depicts the specific binding of the native hormone SRIF14 to human cloned SSTR1-5. The results show the specific binding as IC₅₀ and Ki values. These results generated from the displacement of radiolabeled [¹²⁵I] Tyr SRIF-14 by concentrations ranges of the “cold” SRIF-14. This specific binding data used for calculation of Bmax values which is the concentration of each of the expressed cloned receptor in its transfected cell system.

TABLE 2
The specific binding of SRIF-14 as the basis for experimental conditions of binding
studies to human cloned somatostatin receptors SSTR-1, 2, 3, 4, and 5.

| | Source | | | Ligand | | |
|---|---|---|---|---|---|---|
| Somatostatin | (Human | Incubation | | [¹²⁵I] | | Bmax |
| Receptor | Recombinant | time | | Tyr¹¹- | Specific | (pmol/mg |
| Subtype | cells) | (hours) | IC₅₀ | SRIF 14 | binding | Protein) |
| SSTR-1 | CHO-S | 2 | 0.53 nM | 0.1 nM | 93% | 5.8 |
| SSTR-2 | CHO-K1 | 4 | 15.4 pM | 0.03 nM | 90% | 11.0 |
| SSTR-3 | CHO-K1 | 2 | 0.2 nM | 0.1 nM | 80% | 5.5 |
| SSTR-4 | Chem-1 | 2 | 0.38 nM | 0.1 nM | 85% | 27.5 |
| SSTR-5 | Chem-1 | 2 | 0.53 nM | 0.13 nM | 90% | 5.2 |

The Results of IC₅₀ values were determined by a non-linear, least squares regression analysis using MathIQ™ (ID Business Solutions Ltd., UK). The inhibition constants (Ki) values were calculated using the equation of Cheng and Prusoff (Cheng, Y., Prusoff, W.H., Biochem. Pharmacol. 22:3099-3108, 1973) using the observed IC₅₀ of the tested compound, the concentration of radioligand employed in the assay, and the historical values for the KD of the ligand (obtained experimentally at Eurofins Panlabs, Inc.). The Hill coefficient (nH), defining the slope of the competitive binding curve, was calculated using MathIQ™.

Table 3 below depicts the IC₅₀, Ki and Hill coefficient values of the tested peptides SEQ NO. 1-6 to the human recombinant somatostatin receptors 1-5 in comparison to the native hormone SRIF-14. The results of table 4 below depict the binding affinities values of SEQ ID NO. 7-28 in a screening mode. The data depicts the displacement (%percentage) of the radiolabeled [¹²⁵I] Tyr¹¹-SRIF-14 by the tested compounds SEQ ID NO. 7-28 at 1 and 10 nM for each of the cloned receptors SSTR1-5.

TABLE 3
IC₅₀ (bold), Ki and Hill coefficient values of the tested SEQ ID NO. 1-6 to the human recombinant
somatostatin receptors hSSTR1-5 in comparison to the native hormone SRIF-14.

| | IC₅₀/Ki/nH (nM) | | | | |
|---|---|---|---|---|---|
| | hSSTR-1 | hSSTR-2 | hSSTR-3 | hSSTR-4 | hSSTR-5 |
| SRIF-14 | 0.53/0.21/0.9 | 0.015/0.009/1.2 | 0.2/0.023/0.8 | 0.38/0.5/1.2 | 0.53/0.24/0.8 |
| SEQ ID NO: 1 | 180/150/0.74 | 390/320/1.25 | 0.43/0.15/1.28 | >1000/>1000/1.24 | 310/170/1.2 |
| SEQ ID NO: 2 | >100 | >500 | 0.73/0.25/1.09 | >1000 | >500 |
| SEQ ID NO: 3 | 310/260/0.86 | 590/310/1.62 | 0.83/0.28/1.15 | >1000/>1000/1.14 | >1000/660/1.57 |
| SEQ ID NO: 4 | >1000/880/0.75 | 56/30/0.88 | 1.0 | >10000 | 140/78/0.9 |
| SEQ ID NO: 5 | 780/640/0.88 | 420/220/0.8 | 0.9 | >10000 | 150/82/0.96 |
| SEQ ID NO: 6 | 310/260/.55 | 210/110/0.89 | 0.9 | >1000/>1000/0.99 | 86/48/.77 |

From the data of table 3 it is seen that the SEQ ID NO: 1-6 are nanomolar ligands of human SSTR3 and they are highly selective somatostatin receptor 3 analogs versus their significant lower affinities to the other SSTRs, which may have many therapeutic benefits for diseases or conditions related to overexpression of SSTR-3.

TABLE 4
Binding of SEQ ID NO. 7-28 to human cloned SSTR1-5 as displacement (%) values
at 10 and 1 nM (screening mode). Significant displacement marked in bold.

| | % displacement of [¹²⁵I] Tyr¹¹-SRIF-14 | | | | |
|---|---|---|---|---|---|
| | of SEQ ID NO. 7-28 at 10 and (1) nM. | | | | |
| | hSSTR-1 | hSSTR-2 | hSSTR-3 | hSSTR-4 | hSSTR-5 |
| SEQ ID NO. 8 | 10(0) | 10(0) | 88(61) | 4(5) | 15(0) |
| SEQ ID NO. 9 | 10(4) | 0(0) | 91(59) | 0(0) | 12(0) |
| SEQ ID NO. 10 | >100 | >500 | 75(27) | >1000 | >500 |
| SEQ ID NO. 11 | 19(2) | 0(0) | 88(47) | 0(0) | 0(0) |
| SEQ ID NO. 12 | 10(0) | 0(0) | 93(64) | 0(0) | 13(0) |
| SEQ ID NO. 13 | 15(9) | 12(0) | 78(36) | 0(0) | 4(0) |
| SEQ ID NO. 14 | n.d | n.d. | 89(46) | n.d. | n.d. |
| SEQ ID NO. 15 | 18(6) | 0(1) | 87(49) | 0(0) | 0(0) |
| SEQ ID NO. 16 | 5(0) | 2(6) | 88(47) | 0(0) | 3(0) |
| SEQ ID NO. 17 | 15(9) | 8(11) | 91(68) | 5(7) | 13(15) |
| SEQ ID NO. 18 | 4(0) | 0(0) | 85(49) | 8(0) | 0(0) |
| SEQ ID NO. 19 | 12(7) | 10(12) | 89(56) | 0(0) | 3(9) |
| SEQ ID NO. 20 | 14(8) | 2(0) | 95(66) | 0(0) | 0(0) |
| SEQ ID NO. 21 | 22(23) | 2(0) | 93(59) | 9(9) | 0(0) |
| SEQ ID NO. 22 | 7(8) | 0(0) | 49(0) | 3(0) | 0(0) |
| SEQ ID NO. 23 | 6(4) | 0(0) | 56(19) | 0(0) | 0(0) |
| SEQ ID NO. 24 | n.d. | n.d. | 88(66) | n.d. | n.d. |
| SEQ ID NO. 25 | 15(1) | 0(0) | 94(65) | 8(5) | 16(6) |
| SEQ ID NO. 26 | 24(0) | 0(0) | 86(53) | 0(0) | 1(0) |
| SEQ ID NO. 27 | 3(0) | 0(0) | 51(12) | 0(0) | 0(0) |
| SEQ ID NO. 28 | 23(12) | 0(0) | 79(28) | 0(0) | 0(0) |

Definitions:
n.d. = not determined

From the data of table 3 it is seen that the SEQ ID NO: 7-28 exhibit high affinity and selectivity to human cloned SSTR3 versus their significant lower affinities to the other SSTRs, which may have many therapeutic benefits for diseases or conditions related to overexpression of SSTR-3.

### Example 2: Comparative In Vivo Disposition Studies with Radiolabeled Backbone Cyclic SSTR-3 Analogs

In this in vivo example, tumor versus kidney uptake of SEQ ID NO: 4 and the effect of the substitution of the arginine in position 2 of SEQ ID NO: 1 with the lysine SEQ ID NO: 2 was evaluated.

The rationale for this substitution was to reduce the charge of the peptide by replacement of the arginine with lysine which is less basic. The approved radioligand somatostatin analog—Lutathera® is known to cause nephrotoxicity due to its amphipathic and cationic nature. Therefore, the addition of arginine to somatostatin sequence can result with increased surface activity, nonspecific binding, off-target adverse drug reactions and increased retention in the kidneys which will hamper is used as a radioligand therapy of cancer.

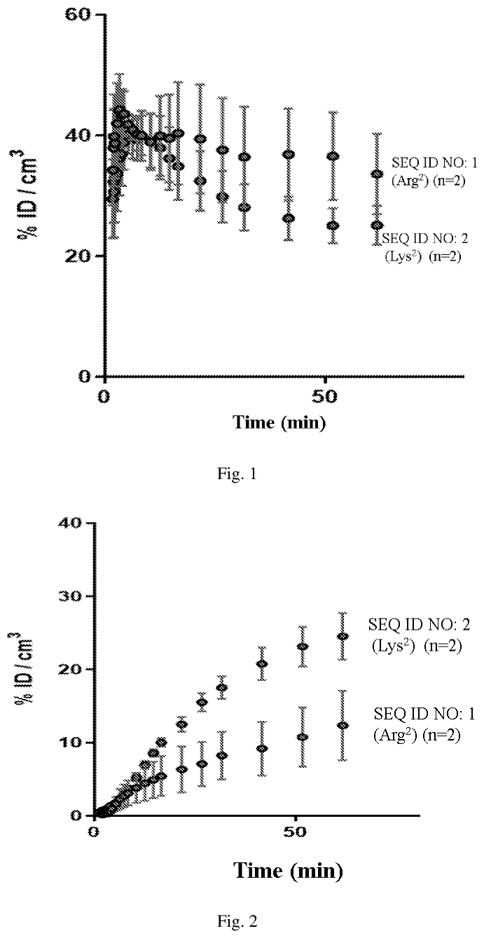

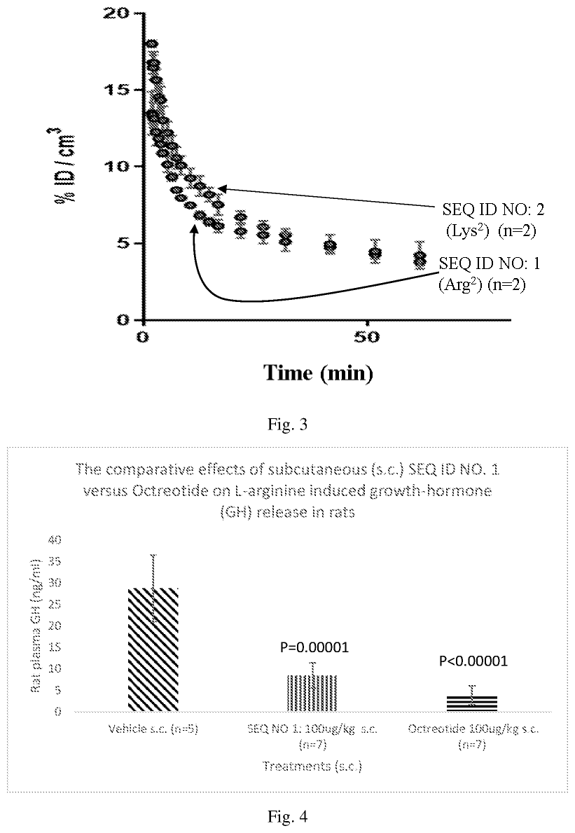

As described in Example 1 above, the successful substitution of the arginine with lysine which preserved the high affinity selectivity of SEQ ID NO: 2 to SSTR-3 (as depicted in table 3) can improve this analog as a better candidate for radioligand therapy and diagnostic. SEQ ID NO: 4 and SEQ ID NO: 10 are the gallium labeled analogs of SEQ ID NO: 1 and SEQ ID NO: 2 respectively. Following their radiolabeling the radioligand were injected to anesthetized naïve mice by intravenous administration. The mice were subjected to PET-CT imaging aimed to assess the disposition profiles of the radioligands. The comparative data of this study showed that radiolabeled SEQ ID NO: 2 exhibited significantly higher clearance of the peptide from the kidneys in comparison to radiolabeled SEQ ID NO: 1 as depicted in the kidneys (FIG. 1) and verified in the bladder imaging (FIG. 2). Under the same experimental conditions both peptides exhibited similar elimination profile from the blood as depicted the kinetic PET imaging of the heart (FIG. 3).

These data support the claim of potential impact of the substitution of the arginine of SEQ ID NO: 1 with the lysine of SEQ ID NO: 2 on the disposition and safety of SEQ ID NO: 2. This PET data confirmed while both peptides had a similar elimination profile from the blood, their disposition in the kidneys and their clearance to urine differ by a significant manner.

### Example 3: Comparative Effects of Somatostatin Analogs on the Release of Growth Hormone, Insulin and Glucagon

Study rationale: The approved drug octreotide is a somatostatin analog with high selectivity to SSTR-2. Octreotide is a potent inhibitor of growth hormone release as well as glucagon and insulin. Therefore, the availability of SSTR2 selective analogs enabled the elucidation of SSTR2 role in the endocrine inhibition of somatostatin of GH, glucagon, and insulin. Octreotide and other approved SSTR2 analogs are indicated for the treatment of acromegaly (over secretion of GH) but with off target effects of insulin and glucagon. The role of SSTR-3 in endocrine effects of GH, glucagon and insulin is unknown due to the lack of nanomolar SSTR3 selective analogs such as SEQ NO. 1 and 2 and their analogs disclosed herein.

In vivo determination of the pharmacodynamic properties of SSTR3 analogs was carried out in rats, according to known procedures (Afargan M. et al., 2001, Pless J. et al 1986).

Inhibition of GH release as a result of peptide administration was measured in Wistar male rats. The analog activity was compared in this study to the SSTR2 approved drug octreotide using 5-8 rats in each treatment group.

Adult male Wistar rats weighing 200-220 g, were maintained on a constant light-dark cycle (light from 8:00 to 20:00 h), temperature (21±3° C.), and relative humidity (55±10%). Laboratory chow and tap water were available ad libitum. All animals in the study were fasted overnight, animals were placed in cages with metal mesh to prevent coprophagia with free access to water. On the day of the experiment, rats were anesthetized with Nembutal (IP, 60 mg/kg). Ten minutes after anesthesia, drugs were administrated S.C. at 100-1000 microgram/kg dose. At 10 minutes after drugs administration the stimulation of GH and glucagon release were carried out by i.v. bolus administration of 0.5 g/kg L-arginine, stimulation of insulin release was carried out in separate animal groups that received i.v. bolus of 0.5 g/kg of glucose. Terminal blood samples in EDTA were collected at 5 minutes after stimulation. Blood samples were and centrifuged immediately. Plasma was separated and kept frozen at −20° C. until assayed.

Rat growth hormone (rGH), glucagon and insulin levels were determined by commercial ELISA kits using the Millipore ELISA kits (Billerica, MA, USA). The ELISA kit for rat plasma GH, Millipore, Cat. No. EZRMGH-45K was performed according to the validated detection method of colorimetric sandwich ELISA protocol (Supriya S. et al., 2013). The ELISA kit for rat plasma insulin, Millipore Cat. No. MMEZRMI13K (Merck) was performed according to the validated detection method of fluorescent colorimetric protocol (Xu, P.Z. et al., 2012). The ELISA kit for rat plasma glucagon, Millipore, Cat. No. EZGLU-35 was performed according to the validated detection method of chemiluminescent sandwich ELISA protocol (Soyeon Y. et al., 2021).

Data analysis of rat plasma GH, glucagon and insulin levels was evaluated by the averaged plasma hormones levels of each group. Statistical comparisons between three or more groups were performed by GraphPad Prism 9, using one-way ANOVA with Tukey's honestly significant difference (HSD) post hoc test. The Tukey's multiple comparison tests were curried at 95% confidence interval. Results are expressed as the mean±SEM.

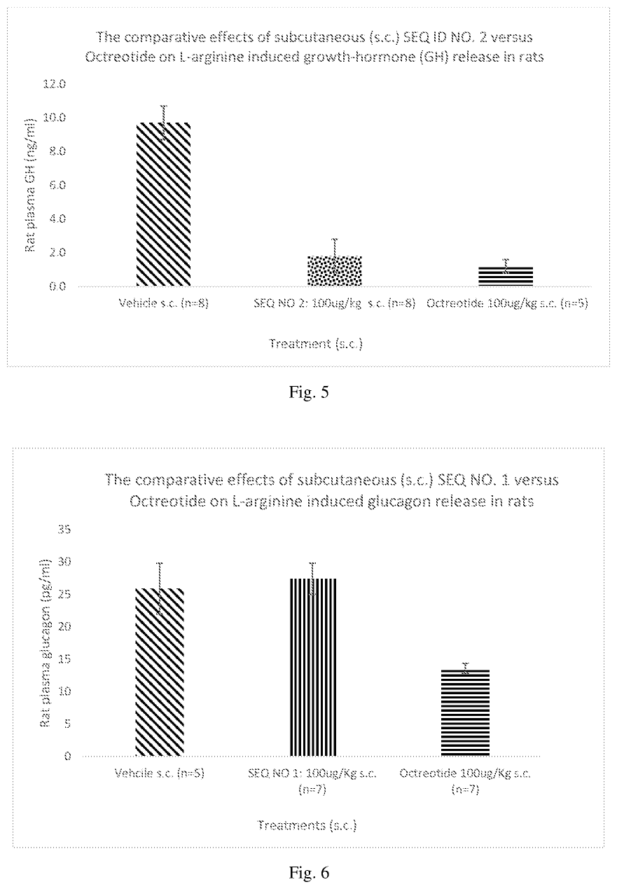

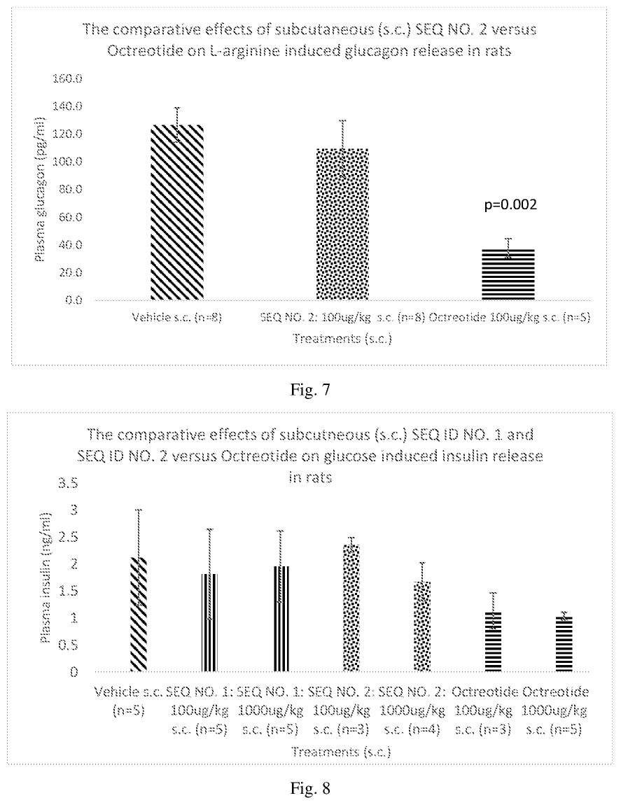

Results: The results of endocrine studies of L-arginine indued GH and glucagon release are depicted in FIGS. 4, 5, 6 and 7. The data show that both nanomolar SSTR3 specific agonists SEQ ID NO.1 and SEQ ID NO.2 are potent inhibitors of pituitary GH release but not of the pancreatic glucagon release. SEQ ID NO.1 and SEQ ID NO.2 elicit significant inhibitory effect of GH release in comparison to approved SSTR2 selective somatostatin agonist octreotide. These results signify that SSTR3 exhibits an anti-secretagogue role of pituitary GH release. It should be emphasized that based on the results of glucagon release, SSTR2 but not SSTR3 is the predominant somatostatin receptor in the pancreas as reported for octreotide, lanreotide, and lutathera. Furthermore, the results of the endocrine studies of glucose induced insulin release show the same trend as observed in glucagon endocrine profile. The data show that both SEQ ID NO.1 and SEQ ID NO.2 did not affect insulin release while octreotide, exhibits anti-secretagogue effect of pancreatic release of insulin (FIG. 8) as reported elsewhere for SSTR2 selective agonists. In summary, these endocrine studies revealed that SSTR3 selective agonists elicit significant anti-secretagogue activity of pituitary GH release but not of pancreatic glucagon or insulin release. These findings support the therapeutic potential of SSTR3 selective agonists in acromegaly and pituitary adenomas. Selective agonists of SSTR3 may have superior on-target activity versus SSTR2 selective agonists with less off-target adverse effects toward the pancreas.

### Example 4: Secondary Pharmacology—Off-Target Interactions for SEQ ID NO. 4. Cellular Agonistic and Antagonistic to 168 Human Cloned GPCRs

It is well known in medicinal chemistry that peptides are considered as relativity flexible molecules. Due to their wide range of chirality of alpha backbone carbons, most peptides adopt more than single conformation in their biospace. This relatively high molecular flexibility makes both endogenous and synthetic peptide-based drugs susceptible for interactions with off-target receptors such as GPCRs that are distinct from their on-target receptor/s. This well-known crosstalk with nonspecific GPCRs reported for many hormones synthetic analogs and especially for somatostatin ligands. This potential nonspecific crosstalk might lead to unexpected adverse effects and therefore it represents one of the main pharmacological concerns in selection of optimal lead compound for drug development. Noteworthy, the hormone somatostatin and its approved synthetic analogs exhibit significant (nanomolar) interactions with off-target GPCRs within the nanomolar range. For example, human hormone cortistatin interacts with all SSTRs and exhibit similar nanomolar affinities to human SSTR1-5 (Avron D.S. et al., 2000, Thomas G. et al., 2018,). The approved somatostatin drug octreotide was reported to exhibit nonspecific binding to opiate and neuromedin receptors (Afargan et al., 2001). The hormone somatostatin and its approved SSTR2 selective analogs octreotide, octreotate (which is the pharmacophre sequence of approved teranogstics DOTATATE, DOTATETOC and lutathatera) and lanreaotide exhibit nanomolar affinities to vasoactive intestinal peptide (VIP) receptors (Irene V. et al., 1994, Orbuch M. et al., 1993). Below is described an evaluation the possible off-target interactions of SEQ ID NO. 4 as a prototype of SSTR3 selective analogs to 168 human cloned GPCRs.

In this example, cellular off-target interactions of both agnostic and antagonstic modes for SEQ ID NO. 4, as a prototype of hSSTR3 selective analogs, against 168 hGPCRs were evaluated using the Eurofins drug discovery panel DiscoveRx. In addition, the off-target interactions of SEQ ID NO. 4 at 1 micromolar overdose which is >1000 above its on-target affinity binding to SSTR3 was determined.

The assays were performed utilizing the PathHunter beta-arrestin enzyme fragment complementation (EFC) technology by Eurofins (CA, USA). The PathHunter® β-Arrestin assay monitors the activation of a GPCR in a homogenous, non-imaging assay format using a technology developed by DiscoveRx called Enzyme Fragment Complementation (EFC) with β-galactosidase ((β-Gal) as the functional reporter. The enzyme is split into two inactive complementary portions (EA for Enzyme Acceptor and ED for Enzyme Donor) expressed as fusion proteins in the cell. EA is fused to β-Arrestin and ED is fused to the GPCR of interest. When the GPCR is activated and β-Arrestin is recruited to the receptor, ED and EA complementation occurs, restoring β-Gal activity which is measured using chemiluminescent PathHunter® Detection Reagents.

Cell Handling: 1. PathHunter cell lines were expanded from freezer stocks according to standard procedures. 2. Cells were seeded in a total volume of 20 μL into white walled, 384-well microplates and incubated at 37° C. for the appropriate time prior to testing. Agonist Format: 1. For agonist determination, cells were incubated with sample to induce response. 2. Intermediate dilution of sample stocks was performed to generate 5× sample in assay buffer. 3. 5 μL of 5× sample was added to cells and incubated at 37° C. or room temperature for 90 or 180 minutes. Final assay vehicle concentration was 1%. Antagonist Format: 1. For antagonist determination, cells were preincubated with antagonist followed by agonist challenge at the EC80 concentration. 2. Intermediate dilution of sample stocks was performed to generate 5× sample in assay buffer. 3. 5 μL of 5× sample was added to cells and incubated at 37° C. or room temperature for 30 minutes. Vehicle concentration was 1%. 4. 5 μL of 6× EC80 agonist in assay buffer was added to the cells and incubated at 37° C. or room temperature for 90 or 180 minutes. Signal Detection: 1. Assay signal was generated through a single addition of 12.5 or 15 μL (50% v/v) of PathHunter Detection reagent cocktail, followed by a one-hour incubation at room temperature. 2. Microplates were read following signal generation with a PerkinElmer Envision™ instrument for chemiluminescent signal detection. Data Analysi: Compound activity was analyzed using CBIS data analysis suite (ChemInnovation, CA). 1. For agonist mode assays, percentage activity was calculated using the following formula: % Activity=100%×(mean RLU of test sample−mean RLU of vehicle control)/(mean MAX control ligand−mean RLU of vehicle control). 2. For antagonist mode assays, percentage inhibition was calculated using the following formula: % Inhibition =100%×(1−(mean RLU of test sample−mean RLU of vehicle control)/(mean RLU of EC80 control−mean RLU of vehicle control)). Results showing an inhibition (or stimulation for assays run in basal conditions) higher than 50% are considered to represent significant effects of the test compounds. 50% is the most common cut-off value for further investigation (determination of IC₅₀ or EC50 values from concentration-response curves) that we would recommend.

Results showing an inhibition (or stimulation) between 25% and 50% are indicative of weak to moderate effects (in most assays, they should be confirmed by further testing as they are within a range where more inter-experimental variability can occur). Results showing an inhibition (or stimulation) lower than 25% are not considered significant and mostly attributable to variability of the signal around the control level.

Low to moderate negative values have no real meaning and are attributable to variability of the signal around the control level. High negative values (≥50%) that are sometimes obtained with high concentrations of test compounds are generally attributable to non-specific effects of the test compounds in the assays. On rare occasion they could suggest an allosteric effect of the test compound.

In vitro cellular screening studies, show that SEQ ID NO.4 exhibited significant selectivity to human SSTR3 only, with no indication of any agonistic or antagonistic interactions of 167 off-target hGPCRs. This data indicates on the novelty and potential therapeutic superiority of SEQ ID NO. 4, and its analogs disclosed herein among known somatostatin ligands as superselective somatostatin SSTR3 agonistic ligands.

TABLE 5
Secondary pharmacology - off-target interactions for SEQ ID NO.
4. Cellular agonistic and antagonistic to 168 human cloned GPCRs.
Secondary pharmacology: Screening for SEQ ID NO. 4 against off-targets
panel of 168 human cloned GPCRs. Agonist screening mode of precent
activity (%) of natural GPCRs ligands in the presence of SEQ ID NO. 4
at 1 μM (>1000 of its on-target IC₅₀ of human SSTR3).

| hGPCR ID | % Activity |
|---|---|
| ADCYAP1R1 | 0 |
| ADORA3 | −6 |
| ADRA1B | −2 |
| ADRA2A | 3 |
| ADRA2B | −1 |
| ADRA2C | −8 |
| ADRB1 | 1 |
| ADRB2 | 0 |
| AGTR1 | −2 |
| AGTRL1 | −1 |
| AVPR1A | 0 |
| AVPR1B | 1 |
| AVPR2 | 0 |
| BDKRB1 | −5 |
| BDKRB2 | 0 |
| BRS3 | 1 |
| C3AR1 | 0 |
| C5aR1 | 0 |
| C5L2 | −1 |
| CALCR (CR) | 0 |
| (CR)L-RAMP1 | −1 |
| (CR)L-RAMP2 | 3 |
| (CR)L-RAMP3 | 0 |
| (CR)R-RAMP2 | 0 |
| (CR)R-RAMP3 | −13 |
| CCKAR | 0 |
| CCKBR | 2 |
| CCR10 | −2 |
| CCR1 | 3 |
| CCR2 | 1 |
| CCR3 | 2 |
| CCR4 | 0 |
| CCR5 | 1 |
| CCR6 | 0 |
| CCR7 | 1 |
| CCR8 | 0 |
| CCR9 | 0 |
| CHRM1 | −1 |
| CHRM2 | 0 |
| CHRM3 | 1 |
| CHRM4 | −3 |
| CHRM5 | 5 |
| CMKLR1 | 0 |
| CNR1 | −1 |
| CNR2 | 2 |
| CRHR1 | 0 |
| CRHR2 | 0 |
| CRTH2 | −1 |
| CX3CR1 | 0 |
| CXCR1 | 0 |
| CXCR2 | −1 |
| CXCR3 | −1 |
| CXCR4 | 3 |
| CXCR5 | 0 |
| CXCR6 | 1 |
| CXCR7 | −1 |
| DRD1 | 0 |
| DRD2L | −1 |
| DRD2S | 0 |
| DRD3 | 9 |
| DRD4 | 0 |
| DRD5 | −2 |
| EBI2 | −1 |
| EDG1 | 0 |
| EDG3 | −1 |
| EDG4 | 6 |
| EDG5 | −2 |
| EDG6 | 10 |
| EDG7 | 0 |
| EDNRA | −1 |
| EDNRB | 0 |
| F2R | 1 |
| F2RL1 | −4 |
| F2RL3 | 2 |
| FFAR1 | −4 |
| FPR1 | −1 |
| FPRL1 | 0 |
| FSHR | 3 |
| GALR1 | 2 |
| GALR2 | −4 |
| GCGR | −2 |
| GHSR | −1 |
| GIPR | −2 |
| GLP1R | 0 |
| GLP2R | −1 |
| GPR1 | 0 |
| GPR103 | 4 |
| GPR109A | 0 |
| GPR109B | −3 |
| GPR119 | 19 |
| GPR120 | −4 |
| GPR35 | 1 |
| GPR92 | 1 |
| GRPR | 0 |
| HCRTR1 | 0 |
| HCRTR2 | 0 |
| HRH1 | −1 |
| HRH2 | −4 |
| HRH3 | −2 |
| HRH4 | −2 |
| HTR1A | 0 |
| HTR1B | −1 |
| HTR1E | 1 |
| HTR1F | 0 |
| HTR2A | −1 |
| HTR2C | 0 |
| HTR5A | 0 |
| KISS1R | 0 |
| LHCGR | 1 |
| LTB4R | −1 |
| MC1R | 1 |
| MC3R | 0 |
| MC4R | 0 |
| MC5R | −5 |
| MCHR1 | −1 |
| MCHR2 | 1 |
| MLNR | 1 |
| MRGPRX1 | 0 |
| MRGPRX2 | 1 |
| MTNR1A | −7 |
| NMBR | 1 |
| NMU1R | 1 |
| NPBWR1 | 2 |
| NPBWR2 | 1 |
| NPFFR1 | −2 |
| NPSR1b | −1 |
| NPY1R | −5 |
| NPY2R | 1 |
| NTSR1 | 3 |
| OPRD1 | −1 |
| OPRK1 | −2 |
| OPRL1 | −3 |
| OPRM1 | 1 |
| OXER1 | −2 |
| OXTR | 0 |
| P2RY1 | −2 |
| P2RY11 | 0 |
| P2RY12 | 1 |
| P2RY2 | 2 |
| P2RY4 | 1 |
| P2RY6 | 0 |
| PPYR1 | 1 |
| PRLHR | 1 |
| PROKR1 | 0 |
| PROKR2 | 2 |
| PTAFR | −2 |
| PTGER2 | 1 |
| PTGER3 | 1 |
| PTGER4 | −2 |
| PTGFR | 0 |
| PTGIR | 0 |
| PTHR1 | 0 |
| PTHR2 | 0 |
| RXFP3 | −3 |
| SCTR | 0 |
| SSTR1 | 32 |
| SSTR2 | 9 |
| SSTR3 | 200 |
| SSTR5 | 16 |
| TACR1 | 0 |
| TACR2 | −1 |
| TACR3 | 1 |
| TBXA2R | 1 |
| TRHR | 3 |
| TSHR(L) | −1 |
| UTR2 | 0 |
| VIPR1 | 0 |
| VIPR2 | −1 |

### Example 5: Secondary Pharmacology—Off-Target Interactions for SEQ ID NO. 3 in Human Cloned GPCR Panel. The Binding and Enzymatic Activity to Off-Target 44 Human Cloned GPCRs

In this example, off-target interactions for SEQ ID NO. 3, as a prototype of hSSTR3 selective analogs, against 44 human cloned GPCRs and GPCR mediated cellular enzymatic activities at 1 micromolar overdose which is >1000 above its on-target affinity binding to SSTR3 were evaluated.

The assays were performed by Eurofins Cerep (Celle 1'Evescault, France). Compound binding was calculated as a % inhibition of the binding of a radioactively labeled ligand specific for each target. Compound enzyme inhibition effect was calculated as a % inhibition of control enzyme activity. In each experiment and if applicable, the respective reference compound was tested concurrently with SEQ ID NO. 3, and the data were compared with historical values determined at Eurofins. The experiment was accepted in accordance with Eurofins validation Standard Operating Procedure. Results showing an inhibition (or stimulation for assays run in basal conditions) higher than 50% are considered to represent significant effects of the test compounds. 50% is the most common cut-off value for further investigation (determination of IC₅₀ or EC₅₀ values from concentration-response curves) that we would recommend.

Results showing an inhibition (or stimulation) between 25% and 50% are indicative of weak to moderate effects (in most assays, they should be confirmed by further testing as they are within a range where more inter-experimental variability can occur).

Results showing an inhibition (or stimulation) lower than 25% are not considered significant and mostly attributable to variability of the signal around the control level.

Low to moderate negative values have no real meaning and are attributable to variability of the signal around the control level. High negative values (>50%) that are sometimes obtained with high concentrations of test compounds are generally attributable to non-specific effects of the test compounds in the assays. On rare occasion they could suggest an allosteric effect of the test compound. The results are expressed as a percent of control specific binding measured specific binding

$$\frac{measured specific binding}{control specific binding} * 100$$

and as a percent inhibition of control specific activity obtained in the presence of SEQ ID NO. 3.

The IC50 values (concentration causing a half-maximal inhibition of control specific activity), EC₅₀ values (concentration producing a half-maximal increase in control basal activity), and Hill coefficients (nH) were determined by non-linear regression analysis of the inhibition/concentration-response curves generated with mean replicate values using Hill equation curve fitting.

$$Y = D + [ \frac{A - D}{1 + {( C / {C}_{50} )}^{nH}} ]$$

where Y=specific activity, A=left asymptote of the curve, D=right asymptote of the curve, C=compound concentration, C50=IC₅₀ or EC₅₀, and nH=slope factor. This analysis was performed using software developed at Cerep (Hill software) and validated by comparison with data generated by the commercial software SigmaPlot® 4.0 for Windows® (©1997 by SPSS Inc.).

$${K}_{i} = \frac{{IC}_{50}}{( 1 + L / {K}_{D} )}$$

where L=concentration of radioligand in the assay, and KD=affinity of the radioligand for the receptor. A scatchard plot is used to determine the KD.

The safety pharmacology panel for SEQ ID NO. 3 shows no interaction with human cloned GPCRs of clinical significance. Moreover, the data shows that known off-target interactions of somatostatin analogs such as the approved drug octreotide with opiate receptors is not indicated for SEQ ID NO. 3 even with overdose of 1 micromolar which is approximately 1000-fold above its on-target IC₅₀ of hSSTR3.

TABLE 6
Secondary pharmacology - off-target interactions
for SEQ ID NO. 3 human cloned GPCR panel. Binding and
enzymatic activity to 44 human cloned off-target GPCRs.
The molecular interactions for SEQ ID NO. 3, at 1
micromolar (>1000-fold its SSTR3 on-target IC50) to
human off-targets (as % displacement of specific
radioligands, or enzymatic activity).

| | |
|---|---|
| | % Activity: |
| | Inhibition of |
| | Control Specific |
| Human molecular target ID (human cloned receptors) | Binding |

Binding

| | |
|---|---|
| A2A (h) (agonist radioligand) | 0 |
| alpha 1A (h) (antagonist radioligand) | −1 |
| alpha 2A (h) (antagonist radioligand) | 2 |
| beta 1 (h) (agonist radioligand) | 5 |
| beta 2 (h) (antagonist radioligand) | −6 |
| BZD (central) (agonist radioligand) | −7 |
| CB1 (h) (agonist radioligand) | 1 |
| CB2 (h) (agonist radioligand) | −1 |
| CCK1 (CCKA) (h) (agonist radioligand) | −15 |
| D1 (h) (antagonist radioligand) | −1 |
| D2S (h) (agonist radioligand) | 3 |
| ETA (h) (agonist radioligand) | −3 |
| NMDA (antagonist radioligand) | 10 |
| H1 (h) (antagonist radioligand) | 7 |
| H2 (h) (antagonist radioligand) | −7 |
| MAO-A (antagonist radioligand) | 3 |
| M1 (h) (antagonist radioligand) | −3 |
| M2 (h) (antagonist radioligand) | −4 |
| M3 (h) (antagonist radioligand) | −7 |
| N neuronal alpha 4beta 2 (h) (agonist radioligand) | −4 |
| delta (DOP) (h) (agonist radioligand) | 10 |
| kappa (h) (KOP) (agonist radioligand) | 10 |
| mu (MOP) (h) (agonist radioligand) | 8 |
| 5-HT1A (h) (agonist radioligand) | −3 |
| 5-HT1B (h) (antagonist radioligand) | 1 |
| 5-HT2A (h) (agonist radioligand) | −2 |
| 5-HT2B (h) (agonist radioligand) | −31 |
| 5-HT3 (h) (antagonist radioligand) | 1 |
| GR (h) (agonist radioligand) | −10 |
| AR(h) (agonist radioligand) | −9 |
| V1a (h) (agonist radioligand) | 0 |
| Ca2+ channel (L, dihydropyridine site) (antagonist | −10 |
| radioligand) | |
| Potassium Channel hERG (human)- [3H] Dofetilide | −9 |
| KV channel (antagonist radioligand) | −5 |
| Na+ channel (site 2) (antagonist radioligand) | 3 |
| norepinephrine transporter (h) (antagonist | −10 |
| radioligand) | |
| dopamine transporter (h) (antagonist radioligand) | 12 |
| 5-HT transporter (h) (antagonist radioligand) | −9 |

Enzymatic

| | |
|---|---|
| COX1(h) | 9 |
| COX2(h) | 18 |
| PDE3A (h) | 0 |
| PDE4D2 (h) | −2 |
| Lck kinase (h) | −4 |
| acetylcholinesterase (h) | 6 |

### Example 6: Primary Pharmacology—Comparative on-Target Cellular Agonistic Activity for SSTR3 Selective SEQ ID NO. 4 Versus the Hormone Somatostatin (SRIF-14) and the Approved SSTR2 Selective Drug DOTATATE

In this example, cellular agonistic activity for SEQ ID NO. 4, as a SSTR3 specific ligand in comparison to human somatostatin hormone SRIF-14 and the SSTR2 specific drug DOTATATE in SSTR3 and SSTR2 transfected CHO cells respectively was evaluated, using the following method: Comparative specific agonist activities, were determined by concentration response for each tested compound for EC₅₀ values. Agonistic activity assessed by ligand induced beta-arrestin mediated receptor internalization as described in example 4.

Comparative studies in CHO cells transfected with hSSTR3 showed that SEQ ID NO. 4 exhibits significant cellular agonistic activity of beta-arrestin mediated SSTR3 internalization. Both SEQ ID NO. 4 and the native hormone SRIF-14 exhibit equal EC₅₀. Surprisingly, the intensity of beta-arrestin response for SEQ ID NO. 4 was exceeded by 2-fold above the native hormone SRIF-14. This increased agonist signal determines SEQ ID NO. 4 as a superagonist of hSSTR3. While under the same experimental conditions, the maximal response for both SRIF-28 and DOTATATE of hSSTR2 agonistic internalization were approximately half of the observed maximal response of SEQ ID NO. 4 of hSTSR3. These comparative cellular responses demonstrate that SEQ ID NO. 4 is a novel (first time known) superagonist of hSSTR3. The results are shown in table 7 below.

TABLE 7
Cellular Internalization EC₅₀ (nM) and Maximal Agonistic
Response Human Cloned SSTR2 and SSTR3 Transfected Cells
(β-Arrestin Luminescence Assay) ^{a}

| | | | Efficacy - | |
|---|---|---|---|---|
| | | | maximal | |
| | | EC₅₀ | response | |
| SSTR | Compound | (nM) | (RLU) | Note |
| SSTR3 | Tyr¹¹-SRIF-14 | 13 | 46680 | Agonist |
| | [^{69/71}Ga]Ga-DOTA-PTR-58 | 16 | 75240 | Super-agonist |
| SSTR2 | SRIF-28 | 3 | 47480 | Agonist |
| | DOTA-TATE | 0.2 | 50080 | Agonist |

Definitions:
^{a} PathHunter ® activated GPCR internalization assays for quantitative measurement (luminescence) of β-Arrestin mediated GPCR internalization. Studies performed by Eurofins Discovery Services.

### Example 7: Comparative On-Target Cellular Internalization of Radiolabeled Compounds

In order to evaluate the cellular internalization of the ¹⁷⁷Lu radiolabeled SEQ ID NO: 6 in comparison to SEQ ID NO: 19 in SSTR3 transfected human cancer cell line to predict the tumor uptake of the radiolabeled compounds, the following example was performed. In addition, the example shows that cellular uptake is a receptor mediated internalization via SSTR3 in SSTR3, and that modified di-Phe enhance tumor uptake.

Comparative SSTR3 receptor mediated internalization, was determined by incubation of the cultivate cancer cells with the ¹⁷⁷Lu radiolabeled compounds and determination of cellular uptake following washes of the cell culture. To evaluate the internalization as a receptor mediated process, the same experiments were performed in the presence of high concentration of cold SSTR3 ligands.

U2OS cells grow as a monolayer at +37° C. in a humidified atmosphere (5% CO2, 95% air). U2OS cancer cells are adherent to plastic flasks. For experimental use, these cancer cells are detached from the culture flask by a 5-minute treatment with trypsin-versene, in Hanks' medium without calcium or magnesium and neutralized by addition of complete culture medium.

The assay was performed as follows: Transfected human osteosarcoma cells SSTR3-tGFP-U2OS were plated in 6 well plate, 24 hours before the assay to have 80-90% confluency at the day of the assay. 30 minutes before the assay, medium was removed, and cells were washed with tempered Dulbecco's Phosphate Buffered Saline (DPBS, Sigma) and fresh medium (250 μL) is added. For total cellular uptake, 20 nM of the radioligand SEQ ID NO: 6 or SEQ ID NO: 19 were diluted in cell medium (250 μL) added to each well, in triplicate (final radioligand concentration: 10 nM). For non-specific binding, 20 μM (1000 in excess) of cold SEQ ID NO: 4 or cold SEQ ID NO: 6 or SEQ ID NO: 1 and 20 nM of ¹⁷⁷Lu-DOTA-PTR-58 radioligand solution in cell medium (250 μL) were added to each well, in triplicate (final cold peptide concentration: 10 μM; final radioligand concentration: 10 nM). The plates were incubated for 4 hours at +37° C. (air containing 5% CO2, humidified atmosphere). At the end of incubation, the plates were placed on ice to stop any internalization process. The medium, containing the free radioligand fraction, was collected and the cells were washed twice (2×750 μL) with ice-cold DPBS. The medium and the PBS washes were collected in the same vial from each well (final volume 1500 μL). The radioactivity of the cell pellets was counted by gamma counter and represents the cellular uptake as percentage of the administrated radioactivity. The radioactivity readouts of the wash-media were used for mass balance.

Comparative studies in human SSTR3 transfected osteosarcoma U2OS cells showed that ¹⁷⁷Lu radiolabeled SEQ ID NO. 6 and 19 exhibit significant cellular internalization of 22 and 30% respectively. The significance of these cellular internalization values is comparable to the reported cellular internalization of the approved drug Lutathera® (¹⁷⁷Lu-DOTATATE), which was approximately 30% at 4 hours, as disclosed by the NDA file (NDA 207800) published by the US Food and Drug Administration. It should be emphasized that the results of significant blocking support the specificity of receptor mediated mechanism of the radioligand-SSTR3 complex. Moreover, the blocking experiments show that either the unlabeled DOTA conjugate SEQ ID NO:3 or the cold ^{69/71}Ga labeled SEQ ID NO:4 or the cold ¹⁷⁵Lu labeled SEQ ID NO: 6 were all able to block the internalization which support the claim for all SSTR3 selective ligands disclosed herein as superagonists and selective of the SSTR3 receptor internalization response. The comparative internalization data of SEQ ID NO: 19 versus SEQ ID NO: 6 support the assumptions of additional di-Phe as R² to enhance tumor uptake. The internalization value of the di-Phe analog SEQ ID NO: 19 was approximately 50% above the observed internalization of SEQ ID NO: 6. The results are shown in Table 8 below.

TABLE 8
Primary pharmacology - on-target receptor mediated agonistic
internalization in human osteosarcoma SSTR3 transfected
cells U2OS. Comparative receptor mediated internalization of
¹⁷⁷Lu radiolabeled SEQ ID NO: 6 and 19 in hSSTR3 transfected
human osteosarcoma cell line U2OS, at 4 h.

| Compound | % Total cellular uptake |
|---|---|
| ¹⁷⁷Lu -SEQ ID NO: 6 | 22.1 ± 0.5 |
| ¹⁷⁷Lu -SEQ ID NO: 6 + SEQ ID NO: 4 | 0.9 ± 0.02 |
| (cold ^{69/71}Ga for blocking) | |
| ¹⁷⁷Lu -SEQ ID NO: 6 | 15.5 ± 0.28 |
| ¹⁷⁷Lu -SEQ ID NO: 6 + SEQ ID NO: 6 | 0.6 ± 0.02 |
| (cold ¹⁷⁵Lu for blocking) | |
| SEQ ID NO: 19 | 30 ± 01.7 |
| SEQ ID NO: 19 + SEQ ID NO: 3 | 8.0 ± 0.5 |
| (unlabeled for blocking) | |

### Example 8: Primary Pharmacology—In Vivo Biodistribution and Tumor Uptake in Rodents Bearing Xenograft of Transfected SSTR3 HEK293 Cells

In this example, imaging performance, biodistribution and tumor uptake of SSTR3 selective radioligands in rodents bearing xenografts of transfected SSTR3 cells was determined using the following method: Animal handling was conducted in accordance with the European Council Directive 2010/63/UE. All experimental procedures were approved by the Institutional Ethical Committee and local authorities. For biodistribution studies, female nude nu/nu Balb/c mice or nude nu/nu rats (Charles River) were used. Dynamic PET-CT imaging experiments were performed up to 240 minutes in mice and up to 270 minutes in rats, under isoflurane anesthesia following the intravenous injection of the radiolabeled tracers. All experiments were performed during the light phase of the light—dark cycle. Biodistribution was determined by quantitative analysis of the PET-CT scan and calculated as % percentage of cm³ of the total injected dose (%ID/g) or by the value of SUV which is the ratio of the image-derived radioactivity concentration C_{img} and the whole-body concentration of the injected radioactivity Cᵢₙⱼ.

Table 9 summarizes the quantitative PET-CT analysis of biodistribution of the ⁶⁸Ga radiolabeled SEQ ID NO: 4. The data show significant tumor uptake of the tracer in both mouse and rat. The ratio of tumor to kidney (tumor/kidneys) in the rat was positive with approximately 50% tumor uptake above the kidneys. This data supports the in vitro binding and selectivity of the SSTR3 ligands as potential theragnostic agents for the diagnosis and treatment of SSTR3 positive tumors.

TABLE 9
Intravenous ⁶⁸Ga -SEQ ID NO: 4 (1.5Mbq/40 μCi)
dynamic/quantitative PET-CT scan in the mouse.
Xenograft of SSTR3 transfected HEK-293 cells.

| | | PET-CT time | | | | | |
|---|---|---|---|---|---|---|---|
| | | 0-3.8 | 3.8-9 | 9-25 | 25-57 | 150 | 240 |
| | | min | min | min | min | min | min |
| Kidneys | % ID/cm³ | 31.9 | 44.0 | 56.0 | 18.0 | 6.0 | 6.6 |
| | SUV | 8.0 | 11.0 | 14.0 | 72.0 | 24.0 | 26.5 |
| Tumor | % ID/cm³ | 1.9 | 2.25 | 3.1 | 4.4 | 9.6 | 12.3 |
| | SUV | 0.5 | 0.56 | 0.77 | 1.1 | 2.4 | 3.1 |

Intravenous ⁶⁸Ga -SEQ ID NO: 4 (1.85Mbq/50 μCi)
quantitative PET-CT scan at 2.5 h in the rat.
Xenograft of SSTR3 transfected HEK-293 cells.

| | | | | |
|---|---|---|---|---|
| Kidneys | % ID/cm³ | 3.2 | SUV | 7.6 |
| Tumor | | 4.7 | | 11.0 |

### Example 9: PET-CT Scan with Radiolabeled ⁶⁸Ga-SEQ ID NO: 4 in a Human Subject. A Representative Imaging Performance, Biodistribution and Tumor Uptake in a Patient with Desmoplastic Small Round Cell Tumor—DSRCT (a Sarcoma Type of Tumor)

In this example, the safety, biodistribution, imaging performance and tumor uptake in patient with sarcoma type DSRCT tumor positive SSTR3 expression was evaluated. Following radiolabeling of the title compound, the eluted solution of the radiolabel tracer was filtered for sterility and injected intravenously in isotonic solution. PET-CT scan was performed at 30- and 120-minutes post injection of the radiolabeled tracer at approximately 600 Mbq. Body temperature, heart rate, and blood pressure were monitored before during and after the PET-CT scan for safety evaluation. The patient was a 37-year-old male, diagnosed for DSRCT 8 years prior. Over the last 8 years, before the current example, the patient was treated with External beam radiation therapy (EBRT), chemotherapy (Irinotecan/temozolomide, melphalan/treosuflan, gemcitabine/docetaxel, pazopanib, topotecan/cyclophosphamide, cabozantinib, densumab), and laminectomy (to resect spine—tumoral foci). A biopsy analysis was performed 3 months before the PET-CT and indicated high mRNA expression of SSTR3.

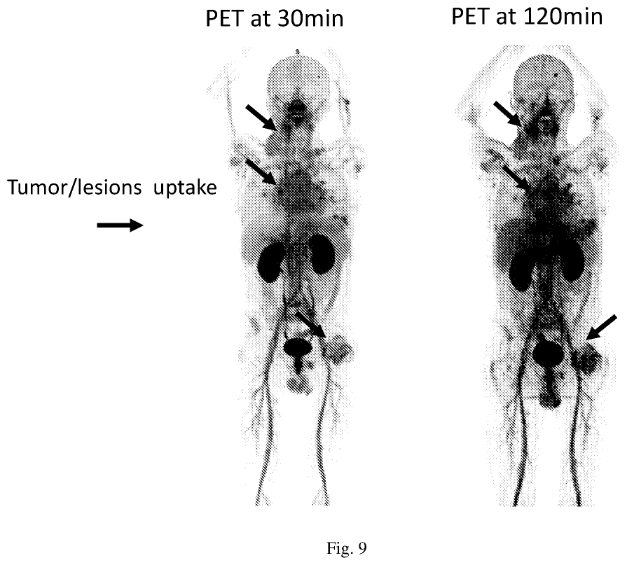

The results of the PET-CT scan for the radiolabeled ⁶⁸Ga-SEQ ID NO: 4 are depicted in FIG. 9. Following the intravenous injection of the ⁶⁸Ga-SEQ ID NO: 4 a rapid elimination of the tracer was observed from the blood to the kidneys. Under these conditions several radioactive foci were detected in the lung, neck and pelvic regions. The observed positive tumor uptake as indicated by the radioactive foci support the prognosis of the tumor and disease prognosis. The biodistribution of the tracer correlated with the high selectivity to SSTR3 with no uptake in off-target organs. These results correlate with the claim for superselective SSTR3 ligand as indicated in the GPCR panel depicted in example 4 herein. The safety evaluations showed no abnormal toxicity signs for the tracer and supported the significant high on-target selectivity of SEQ ID NO: 3.

### Example 10: The Radiolabeling Procedures of ⁶⁸Ga or ¹⁷⁷Lu of DOTA-Peptide Conjugated Disclosed Herein SEQ ID NO: 3, 7, 9, 11, 12, 14, 15, 16, 18-24.

In this example, DOTA-peptide conjugates with radioisotopes ⁶⁸Ga or ¹⁷⁷Lu were prepared, and compatibility, efficiency, purity, analytical identification of the tracer product and reproducibility of the radiolabeling procedures were determined.

The methods described herein are examples of radiolabeling of SEQ ID NO: 3 and 9 but were used as a standard operating procedure of radiolabeling for all DOTA-peptide conjugates disclosed herein. The [⁶⁸Ga]Ga-DOTA-peptide was radiolabeled using the iTM ⁶⁸Ge/⁶⁸Ga a generator (GMP; Isotope Technologies Munich, GmBH, Munich, Germany) and automated module (iQS-TS, Isotope Technologies Munich, GmBH, Munich, Germany) in a GMP-compliant process. In brief, ⁶⁸Ga³⁺ (half-life 68 min; β+ 89%; Eβ+ max. 1.9 MeV) was eluted from the ⁶⁸Ge/⁶⁸Ga radionuclide generator (1850 GBq) to the reaction vessel using 5 mL of hydrochloric acid (0.05 M). The reaction vessel contained 25 micrograms of DOTA-peptide precursor (supplied by Starget Pharma, Ramat-Hasharon, Israel), dissolved in 1 mL of sodium acetate buffer (0.25 M) and was pre-heated to 85° C. The radiolabeling was performed over 5 minutes from to end of the generator elution, using a disposable cassette and labeling kit (Isotope Technologies Munich, GmBH, Munich, Germany). The reaction mixture was then loaded onto a C18 SPE Sep-Pak cartridge (pre-activated using 1.5 mL of ethanol solution followed by 4 mL of 0.9% NaCl solution). The SPE cartridge was subsequently washed with 3 mL of 0.9% NaCl. The final product, [⁶⁸Ga]Ga-DOTA-peptide, was eluted using 1.2 mL of 50% (v/v) ethanol in ultra-pure water followed by 9 mL of 0.9% NaCl, to yield a final volume solution of 10.5-11.5 mL in the final product (5.8% ethanol). The final product was then filter sterilized using a 0.22 μm filter (Cathivex-GV, Darmstadt, Germany) and subjected to quality control analyses in compliance with European Pharmacopoeia. The [⁶⁸Ga]Ga-DOTA-peptide final product solution was visually inspected for being clear and colorless, and obtained in a pH range of 4.0-8.0. The product identity was confirmed using analytical HPLC and compared to the non-radiolabeled [⁶⁸Ga]Ga-DOTA-peptide reference standard (piCHEM, Raaba-Grambach, Austria). The radiochemical purity was confirmed by HPLC and was routinely >93%. The radionuclide identity was confirmed using half-life determination and a gamma spectrum of the main peak. In addition, filter integrity test, endotoxin measurements and sterility tests were performed. Analytical HPLC was performed using Shimadzu analytical HPLC system (‘HPLC 3’) equipped with analytical column (Jupiter 4 μm proteo, 4.6×150 mm, Phenomenex, Torrance, CA, USA) and a UV detector operated at 214 and 240 nm and a radio-detector, Bioscan B-FC 3200 (Eckert & Ziegler Radiopharma, MA, USA). As eluent mixture of HPLC water (A) and acetonitrile (B), both supplemented with 0.1% (v/v) trifluoroacetic acid was used, at a flow rate of 2 mL/min at 40° C., as follow: (t=0) 21% B, (t=8) 25% B, (t=12) 25% B, (t=16) 21% B, (t=20) 21% B. The final product (retention time at 8.1 min) was confirmed using co-elution with the non-radiolabeled reference standard ^{69/71}Ga-DOTA-peptide. The HPLC elution method was design to allow separation of Ga-DOTA-peptide reference standard and the DOTA-peptide precursor. A retention sample was collected and stored for >48 hours, confirmed less than 0.001% of [⁶⁸Ga] content in the original sample. The analytical HPLC conditions were Column: Xselect CSH130 C-18 5 μ 4.6×100mm (ID: AN44). Flow rate: 1,5 mL/min. Mobile phase: 0.1% TFA/water and 0.1% TFA/Acetonitrile. Radioactive detector: Gabi (Raytest). Detector: UV λ=254 nm. Retention time of [¹⁷⁷Lu]-DOTA-peptide: 19-20 min.

For Radiolabeling with ¹⁷⁷Lu, the following procedure was followed: DOTA-peptide: 50 μg lyophilized in a microtube. Water Trace Select. Buffer: Ammonium acetate 0.4 M with Gentisic acid 0.325 M pH 4 (chelexed overnight). ¹⁷⁷LuCl3 in HCl 0.05 M. EDTA 0.1 M. Eppendorf microtube 1.5 mL Low binding. Thermomixer system. Radio-HPLC system: JASCO HPLC system LC-2000 analytical series linked to an UV detector and a Bioscan Flow-Count radioactivity detector ITLC-SA (Agilent) Radio-TLC system: AR-2000 Radio-TLC Imaging Scanner (Bioscan). In a microtube, lutetium-177 ([¹⁷⁷Lu] LuCl3) in HCl 0.05 M was mixed with radiolabeling buffer (ammonium acetate 0.4 M containing gentisic acid 0.325 M, pH 4.1 [2.8× volume of lutetium-177 solution]) and DOTA-peptide (dissolved at 1 mM in water) to reach the targeted specific activity. The reaction mixture was incubated at +90° C. for 30 minutes using a thermomixer system. At the end of the incubation period, the reaction vial was centrifuged. The radiolabeling incorporation was assessed by reversed phase liquid. chromatography and thin layer chromatography: o HPLC-C18 analytical method: Column: Kinetex C18 (2.6 μm, 50×2.1 mm, Phenomenex). Mobile phase: Phase A: water 0.1% TFA. Phase B: MeCN 0.1% TFA. Gradient: 5% mobile phase B to 95% mobile phase B in 5 minutes, back to 5% (B) in 30 s until complete equilibration (5.5 mM). Flow rate: 0.5 miltninute, Detection: radioactivity.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

### REFERENCES

Albertson DG. Gene amplification in cancer. Trends Genet. 2006 Aug.;22(8):447-55. doi: 10.1016/j.tig.2006.06.007. Epub 2006 Jun. 19. PMID: 16787682.
Andreoli E, Suzuki R, Orbaek AW, Bhutani MS, Hauge RH, Adams W, Fleming JB, Barron AR. Preparation and evaluation of polyethyleneimine-single walled carbon nanotube conjugates as vectors for pancreatic cancer treatment. J Mater Chem B. 2014, 7, 2(29), 4740-4747.
Ansquer C, Kraeber-Bodéré F, Chatal JF., Current status and perspectives in peptide receptor radiation therapy. Curr Pharm Des., 2009, 15:2453-62.

Rodrigo Pinheiro Araldi, Diego Grando Módolo, Paulo Luiz de Sá. Júnior, Sílvio Roberto Consonni, Rodrigo Franco de Carvalho, Franco Peppino Roperto, Willy Beçak, Rita de Cassia Stocco, Genetics and metabolic deregulation following cancer initiation: A world to explore, Biomedicine & Pharmacotherapy, Volume 82, 2016, Pages 449-458, ISSN 0753-3322, https://doi.org/10.1016/j.biopha.2016.05.031.
Bloom S.R., Polak J. M. Somatostatin. Br Med J (Clin Res Ed). 1987; 295(6593): 288-290.
Caroline A. Barton, Neville F. Hacker, Susan J. Clark, Philippa M. O'Brien, DNA methylation changes in ovarian cancer: Implications for early diagnosis, prognosis and treatment, Gynecologic Oncology, Volume 109, Issue 1, 2008, Pages 129-139, ISSN 0090-8258, https://doi.org/10.1016/j.ygyno.2007.12.017.
Chin RI, Wu FS, Menda Y, Kim H. Radiopharmaceuticals for Neuroendocrine Tumors. Semin Radiat Oncol. 2021; 31(1):60-70
Cives M, Strosberg J.Radionuclide Therapy for Neuroendocrine Tumors. Curr Oncol Rep. 2017;19(2):9.
Elston MS, Meyer-Rochow GY, Conaglen HM, Clarkson A, Clifton-Bligh RJ, Conaglen JV, et al. Increased SSTR2A and SSTR3 expression in succinated ehydrogenase-deficient pheochromocytomas and paragangliomas. Hum Pathol 2015; 46:390-6.
Eychenne R., Bouvry C., Bourgeois M., Loyer P., Benoist E., Lepareur N. Overview of Radiolabeled Somatostatin Analogs for Cancer Imaging and Therapy. Molecules. 2020; 25(17): 4012.
Florio T, Morini M, Villa V, Arena S, Corsaro A, Thellung S, Culler MD, Pfeffer U, Noonan DM & Schettini G et al. Somatostatin inhibits tumor angiogenesis and growth via somatostatin receptor-3-mediated regulation of endothelial nitric oxide synthase and mitogen-activated protein kinase activities. Endocrinology 2003; 144: 1574-1584.
Haider M, Al-Toubah T, El-Haddad G, Strosberg J. Molecular imaging and radionuclide therapy of neuroendocrine tumors. Curr Opin Endocrinol Diabetes Obes. 2020;27(1): 16-21.
Herrera-Martinez AD, Gahete MD, Pedraza-Arevalo S, Sanchez-San-chez R, Ortega-Salas R, Serrano-Blanch R, et al. Clinical and functional implication of the components of somatostatin system in gastroenteropancreatic neuro endocrine tumors. Endocrine, 2018;59:426-37.
Hofiand LJ, van Koetsveid PM, Waaijers M, Zuyderwijk J, Lamberts SWJ. Relative potencies of the somatostatin analogs octreotide. BIM 23014 and RC-160 on the inhibition of hormone release by cultured human endocrine tumor cells and normal rat anterior pituitary cells. Endocrinology, 1994, 134:301-6.
Hofland LJ, Feelders RA, de Herder WW, Lamberts SW. Pituitary tumours: the sst/D2 receptors as molecular targets. Mol Cell Endocrinol. 2010; 326:89-98.
Hukovic N., Panetta R., Kuma U., Patel Y.C. Agonist-dependent regulation of cloned human somatostatin receptor types 1-5 (hSSTR1-5): subtype selective internalisation or upregulation. Endocrinology. 1996; 137:4046-4049.
Ibanez-Costa A, Rivero-Cortes E,Vazquez-Borrego MC, Gahete MD,Jimenez-Reina L, Venegas-Moreno E, et al. Octreotide and pasireotide(dis)similarly inhibit pituitary tumor cells in vitro. J Endocrinol 2016; 231:135-45.
Ito T, Lee L, Jensen RT. Treatment of symptomatic neuroendocrine tumor syndromes: recent advances and controversies. Expert Opin Pharmacother. 2016;17(16):2191-2205.
Kaur, S., Baine, M. J., Jain, M., Sasson, A. R., & Batra, S. K.,. Early diagnosis of pancreatic cancer: challenges and new developments. Biomarkers in medicine, 2012, 6(5), 597-612.
Kreuzer O J, Krisch B, Déry O, Bunnett N W, Meyerhof W. Agonist-mediated endocytosis of rat somatostatin receptor subtype 3 involves beta-arrestin and clathrin coated vesicles. J Neuroendocrinol. 2001;13(3):279-87.
Kwekkeboom D, Krenning EP, de Jong M. Peptide receptor imaging and therapy. J Nucl Med., 2000, 41:1704-13.
Lee M, Lupp A, Mendoza N, Martin N, Beschorner R, Honegger J, et al. SSTR3 is a putative target for the medical treatment of gonadotroph adenomas of the pituitary. Endocr Relat Cancer 2015; 22:111-9.
Maqsood MH, Tameez Ud Din A, Khan AH. Neuroendocrine Tumor Therapy with Lutetium-177: A Literature Review. Cureus. 2019 30;11(1):e3986.
Mari C. V_azquez-Borrego, Vandana Gupta, Alejandro Ibanez-Costa, Manuel D. Gahete, Eva Venegas-Moreno, Álvaro Toledano-Delgado, David A. Cano, Crist_obal Blanco-Acevedo, Rosa Ortega-Salas, Miguel A. Jap_on, Ana Barrera-Martín, Alexandre Vasiljevic, Jason Hill, Shengwen Zhang, Heather Halem, Juan Solivera, G_erald Raverot, María A. G_alvez, Alfonso Soto-Moreno, Marcelo Paez-Pereda, Michael D. Culler, Justo P. Castano, and Raul M. Luque. A somatostatin receptor subtype-3 (SST3) peptide agonist shows antitumor effects in experimental models of nonfunctioning pituitary tumors. Clin Cancer Res., 2020, 26, 957-69.
Mikolajczak R, Maecke HR. Radiopharmaceuticals for somatostatin receptor imaging. Nucl Med Rev Cent East Eur. 2016;19(2):126-32.
Møller LN, Stidsen CE, Hartmann B, Holst JJ. Somatostatin receptors. Biochim Biophys Acta. 2003; 22;1616(1):1-84.
Negrini, S., Gorgoulis, V. G., & Halazonetis, T. D. Genomic instability—an evolving hallmark of cancer. Nature reviews Molecular cell biology, 2010, 11(3), 220-228.
Oronsky B, Ma PC, Morgensztern D, Carter CA. Nothing But NET: A Review of Neuroendocrine Tumors and Carcinomas. Neoplasia. 2017;19(12):991-1002.
Padma, V. An overview of targeted cancer therapy. BioMed 5, 19, 2015, https://doi.org/10.7603/s40681-015-0019-4
Paganelli G, Zoboli S, Cremonesi M, Bodei L, Ferrari M, Grana C, et al. Receptor-mediated radiotherapy with 90Y-DOTA-D-Phel-Tyr3-octreotide. Eur J Nucl Med Mol Imaging, 2001, 28:426-34.
Qian ZR, Li T, Ter-Minassian M, Yang J, Chan JA, Brais LK, Masugi Y, Thiaglingam A, Brooks N, Nishihara R, Bonnemarie M, Masuda A, Inamura K, Kim SA, Mima K, Sukawa Y, Dou R, Lin X, Christiani DC, Schmidlin F, Fuchs CS, Mahmood U, Ogino S, Kulke MH. Association Between Somatostatin Receptor Expression and Clinical Outcomes in Neuroendocrine Tumors. Pancreas. 2016; 45(10):1386-1393.
Reubi JC, Waser B, Schaer JC, Laissue JA. Somatostatin receptor sst1-sst5 expression in normal and neoplastic human tissues using receptor autoradiography with subtype-selective ligands. Eur J Nucl Med., 2001, 28:836-46.
Scholl, S., Beuzeboc, P., & Pouillart, P. Targeting HER2 in other tumor types. Annals of oncology, 2001, 12, S81-S87.
Schramm, A., De Gregorio, N., Widschwendter, P., Fink, V., & Huober, J. Targeted therapies in HER2-positive breast cancer-a systematic review. Breast care, 2015, 10(3), 173-178.
Strosberg J, Leeuwenkamp O, Siddiqui MK. Peptide receptor radiotherapy re-treatment in patients with progressive neuroendocrine tumors: A systematic review and meta-analysis. Cancer Treat Rev. 2021 Feb; 93:102141.
Sun L, Coy DH. Somatostatin and its Analogs. Curr Drug Targets. 2016;17(5):529-37
Theodoropoulou M & Stalla G.K., Somatostatin receptors: from signaling to clinical practice. Frontiers in Neuroendocrinology; 2013; 34: 228-252.
Tai W, Mo R, Lu Y, Jiang T, Gu Z. Folding graft copolymer with pendant drug segments for co-delivery of anticancer drugs. Biomaterials 2014, 35(25), 7194-203.
Theodoropoulou M, Stalla GK., Somatostatin receptors: from signaling to clinical practice. Front Neuroendocrinol. 2013; 34(3):228-52.
Tower-Gilchrist C, Styers ML, Yoder BK, Berbari NF, Sztul E. Monitoring endosomal trafficking of the G protein-coupled receptor somatostatin receptor 3. Methods Enzymol. 2014; 534:261-80.
Tsimberidou, A. M. Targeted Therapy In Cancer. Cancer chemotherapy and pharmacology, 2015, 76(6), 1113.
Wang, L. Early Diagnosis of Breast Cancer, Sensors, 2017, 17, 1572. https://doi.org/10.3390/s17071572
Yu, J., Cao, F., Zhao, X., Xie, Q., Lu, M., Li, J., Zhi, Y. & Sun, Y., Correlation and

Comparison of Somatostatin Receptor Type 2 Immunohistochemical Scoring Systems with 68Ga-DOTATATE Positron Emission Tomography/Computed Tomography Imaging in Gastroenteropancreatic Neuroendocrine Neoplasms., 2021, Neuroendocrinology. DOI: 10.1159/000517530.
https://www.proteinatlas.org/ENSG00000278195-SSTR3/tissue, last accessed on 9 of Feb., 2022

1-40. (canceled)

41. A method for treatment or diagnosis of a disease associated with cancer associated with overexpression of SSTR3, comprising administering to a patient in need thereof an effective amount of the analog having the formula:
a. R¹-R²-DPhe-R³-Cys-R⁴-DTrp-Lys-Thr-R⁶-R⁵
wherein R¹ is an active agent, or is absent;
R² is a linker, an active agent, or is absent;
R³ is either Arg, Lys, or Orn; or optionally a polypeptide of 3 or two amino acids Glu-Glu-R⁷ or Glu-R⁷, wherein R⁷ is Arg, Lys, or Orn;
R⁴ is either Phe or Tyr; and
R⁵ is a NTAG having as structure of N-ThioAlkyl-Glycine 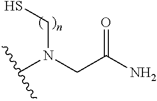 [SMILES: *N(CS)CC(N)=O]
n is the number of methylene groups from 1 to 5,
R⁶ is either Phe or Tyr, wherein optionally a disulfide bond is formed between
R⁵ and the cysteine residue; or a pharmaceutically acceptable salt thereof.

42. The method according to claim 41 wherein the cancer is a sarcoma.

43. The method according to claim 42 wherein the sarcoma is Desmoplastic Small Round Cell Tumor.

44. The method according to claim 41 wherein R¹ is an active agent selected from the group consisting of: an imaging moiety, a therapeutic moiety, a dye, a fluorescent moiety, a toxin, a chelator, a metal atom moiety, a radioactive atom moiety, a nanoparticle, an ethylene glycol polymer, a photosensitizer, a liposome constituent micelle constituent and a tumor targeting moiety, such as a lipid or RGD.

45. The method according to claim 44 wherein R¹ is a chelator.

46. The method according to claim 45 wherein the chelator is selected from the group consisting of: DOTA (1,4,7,10-tetraazacycl ododecane-1,4,7,10-tetraacetic acid), NOTA (2-(4,7-bis(2-(tert-butoxy)-2-oxoethyl)-1,4,7-triazonan-1-yl) acetic acid), NODA (4-(4,7-bis(2-(tert-butoxy)-2-oxoethyl)-1,4,7-triazacyclononan-1-yl)-5-(tert-butoxy)-5-oxopentanoic acid) and EDTA (ethylenediaminetetraacetic acid).

47. The method according to claim 45 further comprising an atom moiety, comprising an atom selected from the group consisting of: iodine-123, iodine-125, iodine-131, fluorine-18, carbon-11, carbon-14, tritium, nitrogen-13, oxygen-15, phosphorous-32, technetium-99m, chromium-51, cobalt-57, cobalt-58, erbium-169, gallium-67, gallium-68, copper-64, indium-111, iron-59, lutetium-175, lutetium-177, radium-223, rubidium-82, samarium-153, selenium-75, strontium-89, thallium-201, yttrium-90, and actinium-225.

48. The method according to claim 47 wherein the atom is gallium-68 or lutetium-177.

49. The method according to claim 45 wherein R² is absent, R³ is arginine, R⁴ is phenylalanine, and R⁶ is phenylalanine.

50. The method according to claim 45 wherein n is 2.

51. The method according to claim 46 wherein R¹ is DOTA (1,4,7,10-tetraazacycl ododecane-1,4,7,10-tetraacetic acid).

52. The method according to claim 51 wherein R² is absent, R³ is arginine, R⁴ is phenylalanine, R⁶ is phenylalanine, and n is 2.

53. The method for diagnosis of a disease, according to claim 52, further comprising subjecting the patient to an imaging procedure.

54. The method for treatment of disease, according to claim 52, wherein the chelator is bound to a radioactive atom moiety.

55. The method according to claim 54, wherein the radioactive atom moiety is actinium-225 or lutetium-177.


SEQUENCE LISTING (28 sequences)

SEQ ID NO: 1 - protein, 9 aa, Artificial Sequence
  Synthetic peptide
  MOD_RES (1)..(1): D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 2 - protein, 9 aa, Artificial Sequence
  Synthetic peptide
  MOD_RES (1)..(1): D-Phenylalanine
  DISULFID (3)..(9)
  MOD_RES (5)..(5): D-Tryptophan
  MOD_RES (9)..(9): N-Thioethyl glycine
XKCFXKTFX

SEQ ID NO: 3 - protein, 9 aa, Artificial Sequence
  Synthetic peptide
  MOD_RES (1)..(1): DOTA covalently bound to D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 4 - protein, 9 aa, Artificial Sequence
  Synthetic peptide
  MOD_RES (1)..(1): gallium-DOTA covalently bound to D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 5 - protein, 9 aa, Artificial Sequence
  Synthetic peptide
  MOD_RES (1)..(1): Copper-DOTA covalently bound to D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 6 - protein, 9 aa, Artificial Sequence
  Synthetic peptide
  MOD_RES (1)..(1): Lutetium-DOTA covalently bound to D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 7 - protein, 9 aa, Artificial Sequence
  Synthetic peptide
  MOD_RES (1)..(1): DOTA-GABA covalently  bound to D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 8 - protein, 9 aa, Artificial Sequence
  Synthetic peptide
  MOD_RES (1)..(1): Gallium-DOTA-GABA covalently bound to D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 9 - protein, 9 aa, Artificial Sequence
  Synthetic peptide
  MOD_RES (1)..(1): DOTA covalently bound to D-Phenylalanine
  DISULFID (3)..(9)
  MOD_RES (5)..(5): D-Tryptophan
  MOD_RES (9)..(9): N-Thioethyl glycine
XKCFXKTFX

SEQ ID NO: 10 - protein, 9 aa, Artificial Sequence
  Synthetic peptide
  MOD_RES (1)..(1): Gallium-DOTA covalently bound to D-Phenylalanine
  DISULFID (3)..(9)
  MOD_RES (5)..(5): D-Tryptophan
  MOD_RES (9)..(9): N-Thioethyl glycine
XKCFXKTFX

SEQ ID NO: 11 - protein, 10 aa, Artificial Sequence
  Synthetic peptide
  MISC_FEATURE (1)..(1): DOTA covalently bound to glycine
  MOD_RES (2)..(2): D-Phenylalanine
  DISULFID (4)..(10)
  MOD_RES (6)..(6): D-Tryptophan
  MOD_RES (10)..(10): N-Thioethyl glycine
GXRCFXKTFX

SEQ ID NO: 12 - protein, 10 aa, Artificial Sequence
  Synthetic peptide
  MISC_FEATURE (1)..(1): DOTA covalently bound to alanine
  MOD_RES (2)..(2): D-Phenylalanine
  DISULFID (4)..(10)
  MOD_RES (6)..(6): D-Tryptophan
  MOD_RES (10)..(10): N-Thioethyl glycine
AXRCFXKTFX

SEQ ID NO: 13 - protein, 12 aa, Artificial Sequence
  Synthetic peptide
  MOD_RES (4)..(4): D-Phenylalanine
  DISULFID (6)..(12)
  MOD_RES (8)..(8): D-Tryptophan
  MOD_RES (12)..(12): N-Thioethyl glycine
RGDXRCFXKTFX

SEQ ID NO: 14 - protein, 12 aa, Artificial Sequence
  Synthetic peptide
  MISC_FEATURE (1)..(1): DOTA covalently bound to arginine
  MOD_RES (4)..(4): D-Phenylalanine
  DISULFID (6)..(12)
  MOD_RES (8)..(8): D-Tryptophan
  MOD_RES (12)..(12): N-Thioethyl glycine
RGDXRCFXKTFX

SEQ ID NO: 15 - protein, 9 aa, Artificial Sequence
  Synthetic peptide
  MOD_RES (1)..(1): DOTA-8-aminooctanoic acid covalently bound to D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 16 - protein, 10 aa, Artificial Sequence
  Synthetic peptide
  MISC_FEATURE (1)..(1): DOTA covalently bound to lysine
  MOD_RES (2)..(2): D-Phenylalanine
  DISULFID (3)..(9)
  MOD_RES (6)..(6): D-Tryptophan
  MOD_RES (10)..(10): N-Thioethyl glycine
KXRCFXKTFX

SEQ ID NO: 17 - protein, 15 aa, Artificial Sequence
  Synthetic peptide
  MISC_FEATURE (1)..(1): DOTA covalently  bound to lysine
  DISULFID (1)..(6)
  MOD_RES (2)..(2): D-Phenylalanine
  MOD_RES (7)..(7): GABA covalently bound to D-Phenylalanine
  DISULFID (9)..(15)
  MOD_RES (11)..(11): D-Tryptophan
  MOD_RES (15)..(15): N-Thioethyl glycine
KXRGDEXKCFXKTFX

SEQ ID NO: 18 - protein, 9 aa, Artificial Sequence
  Synthetic peptide
  MOD_RES (1)..(1): DOTA covalently bound to 4Amb D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 19 - protein, 11 aa, Artificial Sequence
  Synthetic peptide
  MISC_FEATURE (1)..(1): DOTA covalently bound to Phenylalanine
  MOD_RES (3)..(3): D-Phenylalanine
  DISULFID (5)..(11)
  MOD_RES (7)..(7): D-Tryptophan
  MOD_RES (11)..(11): N-Thioethyl glycine
FFXRCFXKTFX

SEQ ID NO: 20 - protein, 10 aa, Artificial Sequence
  Synthetic peptide
  MISC_FEATURE (1)..(1): DOTA covalently bound to Phenylalanine
  MOD_RES (2)..(2): D-Phenylalanine
  DISULFID (4)..(10)
  MOD_RES (6)..(6): D-Tryptophan
  MOD_RES (10)..(10): N-Thioethyl glycine
FXRCFXKTFX

SEQ ID NO: 21 - protein, 9 aa, Artificial Sequence
  Synthetic peptide
  MOD_RES (1)..(1): DOTA covalently bound to D-Phenylalanine
  MOD_RES (2)..(2): Ornithine
  DISULFID (3)..(9)
  MOD_RES (5)..(5): D-Tryptophan
  MOD_RES (9)..(9): N-Thioethyl glycine
XXCFXKTFX

SEQ ID NO: 22 - protein, 11 aa, Artificial Sequence
  Synthetic peptide
  MOD_RES (1)..(1): DOTA covalently bound to D-Phenylalanine
  MOD_RES (4)..(4): Ornithine
  DISULFID (5)..(11)
  MOD_RES (7)..(7): D-Tryptophan
  MOD_RES (11)..(11): N-Thioethyl glycine
XEEXCFXKTFX

SEQ ID NO: 23 - protein, 10 aa, Artificial Sequence
  Synthetic peptide
  MOD_RES (1)..(1): DOTA covalently bound to D-Phenylalanine
  MOD_RES (3)..(3): Ornithine
  DISULFID (4)..(10)
  MOD_RES (6)..(6): D-Tryptophan
  MOD_RES (10)..(10): N-Thioethyl glycine
XEXCFXKTFX

SEQ ID NO: 24 - protein, 9 aa, Artificial Sequence
  Synthetic peptide
  MOD_RES (1)..(1): DOTA covalently bound to D-Phenylalanine
  DISULFID (3)..(9)
  MOD_RES (5)..(5): D-Tryptophan
  MOD_RES (9)..(9): N-Thioethyl glycine
XKCYXKTFX

SEQ ID NO: 25 - protein, 9 aa, Artificial Sequence
  Synthetic peptide
  MOD_RES (1)..(1): DOTA covalently bound to D-Phenylalanine
  DISULFID (3)..(9)
  MOD_RES (5)..(5): D-Tryptophan
  MOD_RES (9)..(9): N-Thioethyl glycine
XRCYXKTFX

SEQ ID NO: 26 - protein, 9 aa, Artificial Sequence
  Synthetic peptide
  MOD_RES (1)..(1): D-Phenylalanine
  DISULFID (3)..(9)
  MOD_RES (5)..(5): D-Tryptophan
  MOD_RES (9)..(9): N-Thioethyl glycine
XRCFXKTYX

SEQ ID NO: 27 - protein, 9 aa, Artificial Sequence
  Synthetic peptide
  MOD_RES (1)..(1): CPT-8-aminooctanoic acid covalently bound to D-Phenylalanine
  DISULFID (3)..(9)
  MOD_RES (5)..(5): D-Tryptophan
  MOD_RES (9)..(9): N-Thioethyl glycine
XRCFXKTFX

SEQ ID NO: 28 - protein, 9 aa, Artificial Sequence
  Synthetic peptide
  MOD_RES (1)..(1): Niraparib--CO(CH2)3CO covalently bound to D-Phenylalanine
  DISULFID (3)..(9)
  MOD_RES (5)..(5): D-Tryptophan
  MOD_RES (9)..(9): N-Thioethyl glycine
XRCFXKTFX